(19) United States
(12) Patent Application Publication
(10) Pub. No.: US 2011/0091955 A1
(43) Pub. Date: Apr. 21, 2011

(54) METHODS AND SYSTEMS FOR TREATING INDUSTRIAL WASTE GASES

(76) Inventors: BRENT R. CONSTANTZ, Portola Valley, CA (US); James Bresson, Los Gatos, CA (US); Divyam Chandra, Santa Clara, CA (US)

(21) Appl. No.: 12/907,933

(22) Filed: Oct. 19, 2010

Related U.S. Application Data
(60) Provisional application No. 61/252,929, filed on Oct. 19, 2009.

(51) Int. Cl. C12P 3/00; C12N 11/14; C12M 1/00; C12N 11/10; C12N 11/08; C12N 11/02

(52) U.S. Cl. 435/168; 435/176; 435/289.1; 435/178; 435/180; 435/177

(57) ABSTRACT

Systems and methods for lowering levels of carbon dioxide and other atmospheric pollutants are provided. Economically viable systems and methods capable of removing vast quantities of carbon dioxide and other atmospheric pollutants from gaseous waste streams and sequestering them in storage stable forms are also discussed.

### CROSS-REFERENCE TO RELATED APPLICATION

This application claims priority to U.S. Provisional Application No. 61/252,929, filed 19 Oct. 2009, which is incorporated herein by reference in its entirety.

### BACKGROUND

The most concentrated point sources of carbon dioxide and atmospheric pollutants (e.g., NOx, SOx, volatile organic compounds (“VOCs”), and particulates) are power plants, particularly power plants that produce power by combusting carbon-based fuels (e.g., coal-fired power plants). Considering that world energy demand is expected to increase, and despite continuing growth in non-carbon-based sources of energy, atmospheric levels of carbon dioxide and other products resulting from combustion of carbon-based fuels are expected to increase as well. As such, power plants utilizing carbon-based fuels are particularly attractive sites for technologies aimed at lowering emissions of carbon dioxide and products resulting from combustion of carbon-based fuels.

Attempts at lowering emissions of carbon dioxide and atmospheric pollutants from power plant waste streams have produced many varied technologies, many of which require very large energy inputs to overcome the energy associated with isolating and concentrating diffuse gaseous species. In addition, current technologies and related equipment may be inefficient and cost prohibitive. As such, it may be desirable to develop an economically viable technology capable of removing vast quantities of carbon dioxide and atmospheric pollutants from gaseous waste streams by sequestering carbon dioxide and atmospheric pollutants in a stable form or by converting it to valuable commodity products.

In consideration of the foregoing, a significant need exists for methods and systems that efficiently and economically sequester carbon dioxide and atmospheric pollutants.

### SUMMARY

In some embodiments, the invention provides, a method comprising (i) contacting a gaseous stream comprising CO₂ with a catalyst to form a solution comprising hydrated CO₂; and (ii) treating the solution to produce a composition comprising a metastable carbonate. In some embodiments, the metastable carbonate is more stable in salt water than in fresh water. In some embodiments, the metastable carbonate is selected from the group consisting of vaterite, aragonite, amorphous calcium carbonate, and combinations thereof. In some embodiments, treating the solution comprises treating the solution comprising hydrated CO₂ with an aqueous solution comprising divalent cations. In some embodiments, the composition comprises calcium carbonate, magnesium carbonate, calcium magnesium carbonate, or a combination thereof. In some embodiments, the composition is further treated to produce a dry particulate composition. In some embodiments, the dry particulate composition has an average particle size of 0.1 to 100 microns. In some embodiments, the dry particulate composition is incorporated into a cement or concrete composition. In some embodiments, the concrete composition further comprises ordinary Portland cement, aggregate, admixture such as supplementary cementitious material, or a combination thereof. In some embodiments, the cement or concrete composition upon combination with water, setting, and hardening has a compressive strength in a range of 20-70 MPa. In some embodiments, the gaseous stream comprises a waste stream or product from an industrial plant selected from power plant, chemical processing plant, or other industrial plant that produces CO₂ as a byproduct. In some embodiments, the catalyst is an enzyme. In some embodiments, treating the solution to produce a composition comprising a metastable carbonate comprises treating the solution with a proton-removing agent. In some embodiments, the method further comprises separating the catalyst from the solution. In some embodiments, the method further comprises producing a building material from the composition comprising the metastable carbonate.

In some embodiments, the invention provides a method comprising (i) contacting a gaseous stream comprising CO₂ with a catalyst to form a solution comprising hydrated CO₂; (ii) treating the solution with a proton-removing agent; and (ii) injecting the solution underground. In some embodiments, the catalyst is an inorganic catalyst, organic catalyst, or a biocatalyst. In some embodiments, the catalyst is carbonic anhydrase. In some embodiments, treating the solution with a proton-removing agent comprises treating the solution with an electrochemically produced proton-removing agent. In some embodiments, the proton-removing agent is sodium hydroxide. In some embodiments, the sodium hydroxide is produced without producing chlorine gas at the anode. In some embodiments, the sodium hydroxide is produced without producing oxygen gas at the anode. In some embodiments, injecting the solution underground comprises injecting the solution into a saline aquifer, a petroleum reservoir, a deep coal seem, a sub-oceanic formation, or some combination thereof. In some embodiments, injecting the solution underground comprises injecting the solution into a saline aquifer. In some embodiments, the capacity of the saline aquifer is increased prior to injecting the solution into the saline aquifer, wherein increasing the capacity of the saline aquifer comprises removing aquifer water.

In some embodiments, the invention provides a composition produced by any of the methods described herein. In some embodiments, the composition comprises an immobilized catalyst on immobilization material, a substrate of the catalyst, a product of the catalyst, and water. In some embodiments, the catalyst is carbonic anhydrase, the substrate is dissolved CO₂, and the product is bicarbonate. In some embodiments, the immobilization material selected from alumina; bentonite; a biopolymers; calcium carbonate; calcium phosphate; carbon; a ceramic support; a clay; a porous metal structure; collagen; glass; hydroxyapatite; an ion-exchange resin; kaolin; a polymer mesh; a polysaccharide; a phenolic polymer; polyaminostyrene; polyacrylamide; poly(acryloyl morpholine); polypropylene; a polymer hydrogel; sephadex; sepharose; a treated silicon oxide; silica gel; and PTFE (polytetrafluoroethylene). In some embodiments, the composition further comprises dissolved SOx, dissolved NOx, one or more dissolved mercury salts, or some combination thereof. In some embodiments, the dissolved SOx comprises sulfite, sulfate, or a combination thereof. In some embodiments, the dissolved NOx comprises nitrite, nitrate, or a combination thereof.

In some embodiment, the invention provides a system comprising a) a source of CO₂; b) a processor comprising a catalyst adapted to produce a solution comprising hydrated CO₂, wherein the processor is operably connected to the source of CO₂; and c) a reactor configured to produce a composition comprising a metastable carbonate. In some embodiments, the system further comprises a source of divalent cations operably connected to the processor and/or the reactor. In some embodiments, the catalyst is immobilized in the processor. In some embodiments, the catalyst is part of an immobilization material selected from alumina; bentonite; a biopolymers; calcium carbonate; calcium phosphate; carbon; a ceramic support; a clay; a porous metal structure; collagen; glass; hydroxyapatite; an ion-exchange resin; kaolin; a polymer mesh; a polysaccharide; a phenolic polymer; polyaminostyrene; polyacrylamide; poly(acryloyl morpholine); polypropylene; a polymer hydrogel; sephadex; sepharose; a treated silicon oxide; silica gel; and PTFE (polytetrafluoroethylene). In some embodiments, the processor comprises a gas-liquid contactor. In some embodiments, the processor comprises a gas-liquid-solid contactor.

### DRAWINGS

The novel features of the invention are set forth with particularity in the appended claims. A better understanding of the features and advantages of the invention will be obtained by reference to the following detailed description that sets forth illustrative embodiments, in which the principles of the invention are utilized, and the accompanying drawings of which:

FIG. 1 provides an illustrative embodiment of a method for producing precipitation material.

FIG. 2 provides an illustrative embodiment of a system for producing precipitation material.

FIG. 3 provides an illustrative embodiment of a gas-liquid-solid contactor.

FIG. 4 provides an illustrative embodiment of a gas-liquid or gas-liquid-solid contactor.

FIG. 5 provides an illustrative embodiment of an end-on view of a gas-liquid or gas-liquid-solid contactor similarly configured as the contactor in FIG. 4.

FIG. 6 provides an illustrative embodiment of a gas-liquid-solid contactor.

FIG. 7 provides an illustrative plot of percentage of CO₂ absorbed over time with a catalyst of the invention.

FIG. 8 provides an illustrative plot of percentage of CO₂ absorbed over time with a catalyst of the invention.

FIG. 9 provides an illustrative plot of percentage of CO₂ absorbed over time with a catalyst of the invention.

### DESCRIPTION

Disclosed herein are methods and systems and compositions derived therefrom, using a source of CO₂, a catalyst optionally in the presence of a proton-removing agent, and a source of divalent cations optionally in the presence of a proton-removing agent, to form compositions of the invention. The compositions formed using the methods and systems of the invention include a CO₂ sequestering component. The compositions disclosed herein include bicarbonates, carbonates, or combinations thereof. The bicarbonates and/or the carbonates may include calcium, magnesium, or combinations thereof. The carbonates include, but are not limited to, vaterite (CaCO₃), amorphous calcium carbonate (CaCO₃.nH₂O), aragonite (CaCO₃), calcite (CaCO₃), ikaite (CaCO₃.6H₂O), a precursor phase of vaterite, a precursor phase of aragonite, an intermediary phase that is less stable than calcite, polymorphic forms in between these polymorphs, or combinations thereof. In some embodiments, the compositions disclosed herein include metastable carbonates including, but not limited to, vaterite (CaCO₃), amorphous calcium carbonate (CaCO₃.nH₂O), aragonite (CaCO₃), ikaite (CaCO₃.6H₂O), a precursor phase of vaterite, a precursor phase of aragonite, an intermediary phase that is less stable than calcite, polymorphic forms in between these polymorphs, or a combination thereof. The compositions disclosed herein may be cementitious compositions which may be hydraulic cement and/or supplementary cementitious material.

The catalyst, used in the methods and systems disclosed herein, includes any organic catalyst, inorganic catalyst, or biocatalyst capable of hydrating CO₂ in the aqueous solution and converting to carbonic acid, bicarbonate, and/or carbonate ions. Such a catalyst may reduce or eliminate the need for a synthetic base or a proton-removing agent, the production of which may be an energy intensive process (e.g., chlor-alkali process). For example, sodium hydroxide produced by a chloralkali process may be an energy intensive process.

In some embodiments, cement compositions of the invention are produced without calcination, thereby reducing the overall CO₂ emission during the process. The methods and systems provided herein, may reduce the carbon footprint by using the carbon dioxide emitted from the power plants or other industrial sources and by sequestering them into the compositions of the invention. The methods and systems provided herein may also reduce the carbon footprint by using the catalyst to hydrate the CO₂ into the aqueous solution. The use of the catalyst to remove the protons from dissolved CO₂ may reduce or eliminate the need for a proton-removing agent. Further, the compositions provided herein may reduce the carbon footprint of cement compositions by partially or completely replacing the carbon-emitting cements such as ordinary Portland cement (OPC) in cement compositions. The compositions of the invention may be mixed with OPC to give the cement composition with an equal or higher strength, thereby reducing the amount of OPC to make cement. For applications such as concrete, compositions of the invention, including cement compositions of the invention, may be mixed with, for example, aggregate, admixtures, or combinations thereof. Aggregate may be prepared according to WO 2009/146436, which was published 3 Dec. 2009, and which is incorporated herein in its entirety.

Before the invention is described in greater detail, it is to be understood that the invention is not limited to particular embodiments described herein as such embodiments may vary. It is also to be understood that the terminology used herein is for the purpose of describing particular embodiments only, and is not intended to be limiting, since the scope of the invention will be limited only by the appended claims. Unless defined otherwise, all technical and scientific terms used herein have the same meaning as commonly understood by one of ordinary skill in the art to which this invention belongs.

It is noted that, as used herein and in the appended claims, the singular forms “a,” “an,” and “the” include plural references unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the invention. Any recited method can be carried out in the order of events recited or in any other order, which is logically possible. Although any methods and materials similar or equivalent to those described herein may also be used in the practice or testing of the invention, representative illustrative methods and materials are now described. Aspects of the invention include methods of preparing a precipitation material or a composition including carbonates, bicarbonates, or combinations thereof. The precipitation material may be produced with a source of CO₂, a source of proton-removing agents (and/or methods of effecting proton removal), a source of divalent cations, and a catalyst, each of which materials are described herein. In some aspects, there are provided methods and systems including contacting a source of CO₂ such as a gaseous stream including CO₂ with a catalyst to form a solution including hydrated CO₂; and treating the solution to produce a composition including a metastable carbonate. Such compositions are described herein.

### Carbon Dioxide

In some embodiments, methods provided herein include contacting a source of CO₂ such as a gaseous stream including CO₂ with a catalyst to form a solution including hydrated CO₂. The catalysts are as described herein. Examples of gaseous streams including CO₂ are described herein. As used herein, the “aqueous solution including hydrated CO₂” includes any form of hydrated CO₂. The hydrated forms of CO₂ include, but are not limited to, carbonic acid, bicarbonate (HCO₃⁻), carbonate (CO₃²⁻), or a combination thereof.

In some embodiments, the source of CO₂ such as a gaseous stream including CO₂ is treated with a catalyst optionally in the presence of a proton-removing agent. The proton-removing agent may adjust the pH of the solution to result in carbonate and/or bicarbonate formation. In some embodiments, the aqueous solution including hydrated CO₂ may be treated with a proton-removing agent to result in the conversion of bicarbonate to carbonate.

In some embodiments, the methods provided herein include treating the solution including the hydrated CO₂ with a source of divalent cations to produce a composition of the invention. In some embodiments, the gaseous stream including CO₂ is contacted with the catalyst and the source of divalent cations simultaneously, to produce the composition of the invention. In some embodiments, the gaseous stream including CO₂ is contacted with the source of divalent cations before the solution is treated with the catalyst, to produce the composition of the invention. In some embodiments, the solution including the hydrated CO₂ is contacted with the source of divalent cations after the CO₂ is treated with the catalyst, to produce the composition of the invention.

In some embodiments, the methods of the invention include contacting a volume of an aqueous solution of divalent cations with a source of CO₂, then subjecting the resultant solution to conditions that facilitate precipitation. Methods of the invention further include contacting a volume of an aqueous solution of divalent cations with a source of CO₂ while subjecting the aqueous solution to conditions that facilitate precipitation. There may be sufficient carbon dioxide in the divalent cation-containing solution to precipitate significant amounts of carbonate- and/or bicarbonate-containing precipitation material (e.g., from seawater); however, additional carbon dioxide may be used.

As used herein, the “source of CO₂” includes any convenient CO₂ source. The CO₂ source may be a gas, a liquid, a solid (e.g., dry ice), a supercritical fluid, or CO₂ dissolved in a liquid. In some embodiments, the CO₂ source is a gaseous CO₂ source. The gaseous stream may be substantially pure CO₂ or comprise multiple components that include CO₂ and one or more additional gases and/or other substances such as ash and other particulates. In some embodiments, the gaseous CO₂ source is a waste gas stream (i.e., a by-product of an active process of the industrial plant) such as exhaust from an industrial plant. The nature of the industrial plant may vary, the industrial plants including, but not limited to, power plants, chemical processing plants, mechanical processing plants, refineries, cement plants, steel plants, and other industrial plants that produce CO as a by-product of fuel combustion or another processing step (such as calcination by a cement plant).

Waste gas streams comprising CO₂ include both reducing (e.g., syngas, shifted syngas, natural gas, hydrogen and the like) and oxidizing condition streams (e.g., flue gases from combustion). Particular waste gas streams that may be convenient for the invention include oxygen-containing combustion industrial plant flue gas (e.g., from coal or another carbon-based fuel with little or no pretreatment of the flue gas), turbo charged boiler product gas, coal gasification product gas, shifted coal gasification product gas, anaerobic digester product gas, wellhead natural gas stream, reformed natural gas or methane hydrates, and the like. Combustion gas from any convenient source may be used in methods and systems of the invention. In some embodiments, combustion gases in post-combustion effluent stacks of industrial plants such as power plants, cement plants, and coal processing plants is used. Thus, the waste streams may be produced from a variety of different types of industrial plants.

Suitable waste streams for the invention include waste streams produced by industrial plants that combust fossil fuels (e.g., coal, oil, natural gas) and anthropogenic fuel products of naturally occurring organic fuel deposits (e.g., tar sands, heavy oil, oil shale, etc.). In some embodiments, a waste stream suitable for systems and methods of the invention is sourced from a coal-fired power plant, such as a pulverized coal power plant, a supercritical coal power plant, a mass burn coal power plant, a fluidized bed coal power plant. In some embodiments, the waste stream is sourced from gas or oil-fired boiler and steam turbine power plants, gas or oil-fired boiler simple cycle gas turbine power plants, or gas or oil-fired boiler combined cycle gas turbine power plants. In some embodiments, waste streams produced by power plants that combust syngas (i.e., gas that is produced by the gasification of organic matter, for example, coal, biomass, etc.) are used. In some embodiments, waste streams from integrated gasification combined cycle (IGCC) plants are used. In some embodiments, waste streams produced by Heat Recovery Steam Generator (HRSG) plants are used in accordance with systems and methods of the invention.

Waste streams produced by cement plants are also suitable for systems and methods of the invention. Cement plant waste streams include waste streams from both wet process and dry process plants, which plants may employ shaft kilns or rotary kilns, and may include pre-calciners. These industrial plants may each burn a single fuel, or may burn two or more fuels sequentially or simultaneously. Other industrial plants such as smelters and refineries are also useful sources of waste streams that include carbon dioxide.

Industrial waste gas streams may contain carbon dioxide as the primary non-air derived component, or may, especially in the case of coal-fired power plants, contain additional components such as nitrogen oxides (NOx), sulfur oxides (SOx), and one or more additional gases. Additional gases and other components may include CO, mercury and other heavy metals, and dust particles (e.g., from calcining and combustion processes). Additional components in the gas stream may also include halides such as hydrogen chloride and hydrogen fluoride; particulate matter such as fly ash, dusts, and metals including arsenic, beryllium, boron, cadmium, chromium, chromium VI, cobalt, lead, manganese, mercury, molybdenum, selenium, strontium, thallium, and vanadium; and organics such as hydrocarbons, dioxins, and PAH compounds.

Suitable gaseous waste streams that may be treated have, in some embodiments, CO₂ present in amounts of 200 ppm to 1,000,000 ppm; or 200 ppm to 500,000 ppm; or 200 ppm to 100,000 ppm; or 200 ppm to 10,000; or 200 ppm to 5,000 ppm; or 200 ppm to 2000 ppm; or 200 ppm to 1000 ppm; or 200 to 500 ppm; or 500 ppm to 1,000,000 ppm; or 500 ppm to 500,000 ppm; or 500 ppm to 100,000 ppm; or 500 ppm to 10,000; or 500 ppm to 5,000 ppm; or 500 ppm to 2000 ppm; or 500 ppm to 1000 ppm; or 1000 ppm to 1,000,000 ppm; or 1000 ppm to 500,000 ppm; or 1000 ppm to 100,000 ppm; or 1000 ppm to 10,000; or 1000 ppm to 5,000 ppm; or 1000 ppm to 2000 ppm; or 2000 ppm to 1,000,000 ppm; or 2000 ppm to 500,000 ppm; or 2000 ppm to 100,000 ppm; or 2000 ppm to 10,000; or 2000 ppm to 5,000 ppm; or 2000 ppm to 3000 ppm; or 5000 ppm to 1,000,000 ppm; or 5000 ppm to 500,000 ppm; or 5000 ppm to 100,000 ppm; or 5000 ppm to 10,000; or 10,000 ppm to 1,000,000 ppm; or 10,00 ppm to 500,000 ppm; or 10,000 ppm to 100,000 ppm; or 50,000 ppm to 1,000,000 ppm; or 50,000 ppm to 500,000 ppm; or 50,000 ppm to 100,000 ppm; or 100,000 ppm to 1,000,000 ppm; or 100,000 ppm to 500,000 ppm; or 200,000 ppm to 1000 ppm, including 200,000 ppm to 2000 ppm, for example 180,000 ppm to 2000 ppm, or 180,000 ppm to 5000 ppm, also including 180,000 ppm to 10,000 ppm.

The waste streams, particularly various waste streams of combustion gas, may include one or more additional components, for example only, water, NOx (mononitrogen oxides: NO and NO₂), SOx (monosulfur oxides: SO, SO₂ and SO₃), VOC (volatile organic compounds), heavy metals such as, but not limited to, mercury, and particulate matter (particles of solid or liquid suspended in a gas). Flue gas temperature may also vary. In some embodiments, the temperature of the flue gas comprising CO₂ is from 0° C. to 2000° C., or 0° C. to 1000° C., or 0° C. to 500° C., or 0° C. to 100° C., or 0° C. to 50° C., or 10° C. to 2000° C., or 10° C. to 1000° C., or 10° C. to 500° C., or 10° C. to 100° C., or 10° C. to 50° C., or 50° C. to 2000° C., or 50° C. to 1000° C., or 50° C. to 500° C., or 50° C. to 100° C., or 100° C. to 2000° C., or 100° C. to 1000° C., or 100° C. to 500° C., or 500° C. to 2000° C., or 500° C. to 1000° C., or 500° C. to 800° C., or such as from 60° C. to 700° C., and including 100° C. to 400° C.

In some embodiments, one or more additional components or co-products (i.e., products produced from other starting materials (e.g., SOx, NOx, etc.) under the same conditions employed to convert CO₂ into carbonates and/or bicarbonates) are precipitated or trapped in precipitation material formed by contacting the waste gas stream comprising these additional components with an aqueous solution comprising divalent cations (e.g., alkaline earth metal ions such as, but not limited to, Ca²⁺ and Mg²⁺), which aqueous solution may further comprise catalyst (e.g., an enzyme such as carbonic anhydrase). In addition, CaCO₃, MgCO₃, and related compounds may be formed without additional release of CO₂. Sulfates, sulfites, and the like of calcium and/or magnesium may be precipitated or trapped in precipitation material (further comprising, for example, calcium and/or magnesium carbonates) produced from waste gas streams comprising SOx (e.g., SO₂). Magnesium and calcium may react to form MgSO₄, CaSO₄, respectively, as well as other magnesium-containing and calcium-containing compounds (e.g., sulfites), effectively removing sulfur from the flue gas stream without a desulfurization step such as flue gas desulfurization (“FGD”). In such embodiments of the invention, catalysts (e.g., carbonic anhydrase) are used in gas-liquid contacting step to catalytically hydrate CO₂ in the presence of SOx, and optionally, NOx and other criteria pollutants. In instances where the aqueous solution of divalent cations contains high levels of sulfur compounds (e.g., sulfate), the aqueous solution may be enriched with calcium and magnesium so that calcium and magnesium are available to form carbonate and/or bicarbonate compounds after, or in addition to, formation of CaSO₄, MgSO₄, and related compounds.

In some embodiments, a desulfurization step may be staged to coincide with precipitation of carbonate- and/or bicarbonate-containing precipitation material, or the desulfurization step may be staged to occur before precipitation. In such embodiments of the invention, catalysts (e.g., carbonic anhydrase) are used in gas-liquid contacting step to catalytically hydrate CO₂ in the absence of SOx, or in the presence of very low levels of SOx. In some embodiments, multiple reaction products (e.g., MgCO₃, CaCO₃, CaSO₄, mixtures of the foregoing, and the like) are collected at different stages, while in other embodiments a single reaction product (e.g., precipitation material comprising carbonates, bicarbonates, sulfates, etc.) is collected. In step with these embodiments, other components, such as heavy metals (e.g., mercury, mercury salts, mercury-containing compounds), may be trapped in the carbonate- and/or bicarbonate-containing precipitation material or may precipitate separately.

A portion of the gaseous waste stream (i.e., not the entire gaseous waste stream) from an industrial plant may be used to produce precipitation material. In some embodiments, the portion of the gaseous waste stream that is employed in precipitation of precipitation material may be 95% or less; or 85% or less; or 75% or less; or 65% or less; or 55% or less; or 45% or less; or 35% or less; or 25% or less; or 15% or less; or 5% or less; or 5% or more; or 15% or more; or 25% or more; or 35% or more; or 45% or more; or 55% or more; or 65% or more; or 75% or more; or 85% or more; or 95%; or between 5-95%; or between 10-95%; or between 20-95%; or between 30-95%; or between 40-95%; or between 50-95%; or between 60-95%; or between 70-95%; or between 80-95%; or between 90-95%; or between 5-75%; or between 10-75%; or between 20-75%; or between 30-75%; or between 40-75%; or between 50-75%; or between 60-75%; or between 70-75%; or between 5-60%; or between 10-60%; or between 20-60%; or between 30-60%; or between 40-60%; or between 50-60%; or between 5-50%; or between 10-50%; or between 20-50%; or between 30-50%; or between 40-50%. In these embodiments, the portion of the gaseous waste stream that is employed in precipitation of precipitation material may be 75% or less, such as 60% or less, and including 50% and less of the gaseous waste stream. In yet other embodiments, substantially (e.g., 80% or more) or the entire gaseous waste stream produced by the industrial plant is employed in precipitation of precipitation material. In these embodiments, 75% or more, 80% or more, such as 90% or more, including 95% or more, up to 100% of the gaseous waste stream (e.g., flue gas) generated by the source may be employed for precipitation of precipitation material. In consideration of the foregoing, the entire portion of the gaseous waste stream obtained from the industrial plant may be subjected to catalytic conditions such that CO₂ is catalytically hydrated to form carbonic acid, bicarbonates and/or carbonates. Also in consideration of the foregoing, the industrial waste stream obtained from the industrial plant may be split in such a way that a fraction of the waste stream is subjected to non-catalytic conditions and the remainder is subjected to catalytic conditions for hydration of CO₂. In such embodiments, 75% or less, such as 60% or less, and including 50% or less of the gaseous waste stream obtained from the industrial is subjected to catalytic conditions for hydration of CO₂. In other such embodiments, substantially (e.g., 75% or more) the entire gaseous waste stream obtained from the industrial plant is subjected to catalytic hydration of CO₂. In such embodiments, 75% or more, 80% or more, such as 90% or more, including 95% or more, or up to 100% of the gaseous waste stream obtained from the industrial plant is subjected to catalytic hydration of CO₂.

Although industrial waste gas offers a relatively concentrated source of combustion gases, methods and systems of the invention are also applicable to removing combustion gas components from less concentrated sources (e.g., atmospheric air), which contains a much lower concentration of pollutants than, for example, flue gas. Thus, in some embodiments, methods and systems encompass decreasing the concentration of pollutants in atmospheric air by producing a stable precipitation material. In these cases, the concentration of pollutants, e.g., CO₂, in a portion of atmospheric air may be decreased by 10% or more, 20% or more, 30% or more, 40% or more, 50% or more, 60% or more, 70% or more, 80% or more, 90% or more, 95% or more, 99% or more, 99.9% or more, or 99.99%. Such decreases in atmospheric pollutants may be accomplished with yields as described herein, or with higher or lower yields, and may be accomplished in one precipitation step or in a series of precipitation steps.

### Divalent Cations

As used herein, the “source of divalent cations” or “divalent cations” or “aqueous solution including divalent cations,” includes any medium containing alkaline earth metals, such as, but not limited to, calcium, magnesium, strontium, barium, etc. or combinations thereof. In some embodiments, the methods and systems of the invention include contacting a volume of an aqueous solution of divalent cations with a source of CO₂ and/or contacting the volume of an aqueous solution of divalent cations with the aqueous solution produced by contacting the source of CO₂ with the catalyst, and subjecting the resultant solution to conditions that facilitate precipitation. In some embodiments, a volume of an aqueous solution of divalent cations is contacted with a source of CO₂ while subjecting the aqueous solution to conditions that facilitate precipitation. Divalent cations may come from any number of different divalent cation sources depending upon availability at a particular location. Such sources include industrial wastes, seawater, brines, hard waters, rocks and minerals (e.g., lime, periclase, material comprising metal silicates such as serpentine and olivine), and any other suitable source.

In some embodiments, industrial waste streams from various industrial processes provide for convenient sources of divalent cations (as well as in some cases other materials useful in the process, e.g., metal hydroxide). Such waste streams include, but are not limited to, mining wastes; fossil fuel burning ash (e.g., combustion ash such as fly ash, bottom ash, boiler slag); slag (e.g. iron slag, phosphorous slag); cement kiln waste; oil refinery/petrochemical refinery waste (e.g. oil field and methane seam brines); coal seam wastes (e.g. gas production brines and coal seam brine); paper processing waste; water softening waste brine (e.g., ion exchange effluent); silicon processing wastes; agricultural waste; metal finishing waste; high pH textile waste; and caustic sludge. Fossil fuel burning ash, cement kiln dust, and slag, collectively waste sources of metal oxides, further described in U.S. patent application Ser. No. 12/486,692, filed 17 Jun. 2009, the disclosure of which is incorporated herein in its entirety. Any of the divalent cation sources described herein may be mixed and matched for the purpose of the invention. For example, material comprising metal silicates (e.g. serpentine, olivine), which are further described in U.S. patent application Ser. No. 12/501,217, filed 10 Jul. 2009, which application is herein incorporated by reference, may be combined with any of the sources of divalent cations described herein for the purpose of the invention.

In some embodiments, a convenient source of divalent cations for preparation of a carbonate/bicarbonate component (e.g., CO₂-sequestering component) in the composition of the invention is water (e.g., an aqueous solution comprising divalent cations such as seawater or surface brine), which may vary depending upon the particular location at which the invention is practiced. Suitable aqueous solutions of divalent cations that may be used include solutions comprising one or more divalent cations, e.g., alkaline earth metal cations such as, but not limited to, Ca²⁺ and Mg²⁺. In some embodiments, the aqueous source of divalent cations comprises alkaline earth metal cations. In some embodiments, the alkaline earth metal cations include calcium, magnesium, or a mixture thereof.

In some embodiments, the aqueous solution of divalent cations comprises calcium in amounts ranging from 50 to 50,000 ppm; or 50 to 40,000 ppm; or 50 to 20,000 ppm; or 50 to 10,000 ppm; or 50 to 5,000 ppm; or 50 to 1,000 ppm; or 50 to 500 ppm; or 50 to 100 ppm; or 100 to 20,000 ppm; or 100 to 10,000 ppm; or 100 to 5,000 ppm; or 100 to 1,000 ppm; or 100 to 500 ppm; or 500 to 20,000 ppm; or 500 to 10,000 ppm; or 500 to 5,000 ppm; or 500 to 1,000 ppm; or 1,000 to 20,000 ppm; or 1,000 to 10,000 ppm; or 1,000 to 5,000 ppm; or 5,000 to 20,000 ppm; or 5,000 to 10,000 ppm; or 10,000 to 20,000 ppm 200 to 5000 ppm, or 400 to 1000 ppm. In some embodiments, the aqueous solution of divalent cations comprises magnesium in amounts ranging from 50 to 40,000 ppm; or 50 to 20,000 ppm; or 50 to 10,000 ppm; or 50 to 5,000 ppm; or 50 to 1,000 ppm; or 50 to 500 ppm; or 50 to 100 ppm; or 100 to 20,000 ppm; or 100 to 10,000 ppm; or 100 to 5,000 ppm; or 100 to 1,000 ppm; or 100 to 500 ppm; or 500 to 20,000 ppm; or 500 to 10,000 ppm; or 500 to 5,000 ppm; or 500 to 1,000 ppm; or 1,000 to 20,000 ppm; or 1,000 to 10,000 ppm; or 1,000 to 5,000 ppm; or 5,000 to 20,000 ppm; or 5,000 to 10,000 ppm; or 10,000 to 20,000 ppm, 200 to 10,000 ppm, 500 to 5000 ppm, or 500 to 2500 ppm.

In some embodiments, a ratio of calcium to magnesium (Ca:Mg) in the aqueous solution of divalent cations is greater than 1:1; or a ratio of greater than 2:1; or a ratio of greater than 3:1; or a ratio of greater than 4:1; or a ratio of greater than 5:1; or a ratio of greater than 6:1; or a ratio of greater than 7:1; or a ratio of greater than 8:1; or a ratio of greater than 9:1; or a ratio of greater than 10:1; or a ratio of greater than 15:1; or a ratio of greater than 20:1; or a ratio of greater than 30:1; or a ratio of greater than 40:1; or a ratio of greater than 50:1; or a ratio of greater than 60:1; or a ratio of greater than 70:1; or a ratio of greater than 80:1; or a ratio of greater than 90:1; or a ratio of greater than 100:1; or a ratio of greater than 150:1; or a ratio of greater than 200:1; or a ratio of greater than 250:1; or a ratio of greater than 300:1; or a ratio of greater than 350:1; or a ratio of greater than 400:1; or a ratio of greater than 450:1; or a ratio of greater than 500:1; or a ratio of 1:1 to 500:1; or a ratio of 1:1 to 450:1; or a ratio of 1:1 to 400:1; or a ratio of 1:1 to 350:1; or a ratio of 1:1 to 300:1; or a ratio of 1:1 to 250:1; or a ratio of 1:1 to 200:1; or a ratio of 1:1 to 150:1; or a ratio of 1:1 to 100:1; or a ratio of 1:1 to 50:1; or a ratio of 1:1 to 25:1; or a ratio of 1:1 to 10:1; or a ratio of 5:1 to 500:1; or a ratio of 5:1 to 450:1; or a ratio of 5:1 to 400:1; or a ratio of 5:1 to 350:1; or a ratio of 5:1 to 300:1; or a ratio of 5:1 to 250:1; or a ratio of 5:1 to 200:1; or a ratio of 5:1 to 150:1; or a ratio of 5:1 to 100:1; or a ratio of 5:1 to 50:1; or a ratio of 5:1 to 25:1; or a ratio of 5:1 to 10:1; or a ratio of 10:1 to 500:1; or a ratio of 10:1 to 450:1; or a ratio of 10:1 to 400:1; or a ratio of 10:1 to 350:1; or a ratio of 10:1 to 300:1; or a ratio of 10:1 to 250:1; or a ratio of 10:1 to 200:1; or a ratio of 10:1 to 150:1; or a ratio of 10:1 to 100:1; or a ratio of 10:1 to 50:1; or a ratio of 10:1 to 25:1; or a ratio of 20:1 to 500:1; or a ratio of 20:1 to 450:1; or a ratio of 20:1 to 400:1; or a ratio of 20:1 to 350:1; or a ratio of 20:1 to 300:1; or a ratio of 20:1 to 250:1; or a ratio of 20:1 to 200:1; or a ratio of 20:1 to 150:1; or a ratio of 20:1 to 100:1; or a ratio of 20:1 to 50:1; or a ratio of 20:1 to 25:1; or a ratio of 50:1 to 500:1; or a ratio of 50:1 to 450:1; or a ratio of 50:1 to 400:1; or a ratio of 50:1 to 350:1; or a ratio of 50:1 to 300:1; or a ratio of 50:1 to 250:1; or a ratio of 50:1 to 200:1; or a ratio of 50:1 to 150:1; or a ratio of 50:1 to 100:1; or a ratio of 100:1 to 500:1; or a ratio of 100:1 to 450:1; or a ratio of 100:1 to 400:1; or a ratio of 100:1 to 350:1; or a ratio of 100:1 to 300:1; or a ratio of 100:1 to 250:1; or a ratio of 100:1 to 200:1; or a ratio of 100:1 to 150:1; or a ratio of 200:1 to 500:1; or a ratio of 200:1 to 450:1; or a ratio of 200:1 to 400:1; or a ratio of 200:1 to 350:1; or a ratio of 200:1 to 300:1; or a ratio of 200:1 to 250:1; or a ratio of 300:1 to 500:1; or a ratio of 300:1 to 450:1; or a ratio of 300:1 to 400:1; or a ratio of 300:1 to 350:1; or a ratio of 400:1 to 500:1; or a ratio of 400:1 to 450:1; or a ratio of 1:1; or a ratio of 2:1; or a ratio of 3:1; or a ratio of 4:1; or a ratio of 5:1; or a ratio of 6:1; or a ratio of 7:1; or a ratio of 8:1; or a ratio of 9:1; or a ratio of 10:1; or a ratio of 11:1; or a ratio of 15:1; or a ratio of 20:1; or a ratio of 30:1; or a ratio of 40:1; or a ratio of 50:1; or a ratio of 60:1; or a ratio of 70:1; or a ratio of 80:1; or a ratio of 90:1; or a ratio of 100:1; or a ratio of 150:1; or a ratio of 200:1; or a ratio of 250:1; or a ratio of 300:1; or a ratio of 350:1; or a ratio of 400:1; or a ratio of 450:1; or a ratio of 500:1. In some embodiments, the ratio of calcium to magnesium (Ca:Mg) is between 2:1 to 5:1, or greater than 4:1, or 4:1. In some embodiments, the ratios herein are molar ratios or weight (e.g., grams, mg, or ppm) ratios.

In some embodiments, where Ca²⁺ and Mg²⁺ are both present, the ratio of Ca²⁺ to Mg²⁺ (i.e., Ca²⁺:Mg²⁺) in the aqueous solution of divalent cations is between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. For example, in some embodiments, the ratio of Ca²⁺ to Mg²⁺ in the aqueous solution of divalent cations is between 1:1 and 1:10; 1:5 and 1:25; 1:10 and 1:50; 1:25 and 1:100; 1:50 and 1:500; or 1:100 and 1:1000. In some embodiments, the ratio of Mg²⁺ to Ca²⁺ (i.e., Mg²⁺:Ca²⁺) in the aqueous solution of divalent cations is between 1:1 and 1:2.5; 1:2.5 and 1:5; 1:5 and 1:10; 1:10 and 1:25; 1:25 and 1:50; 1:50 and 1:100; 1:100 and 1:150; 1:150 and 1:200; 1:200 and 1:250; 1:250 and 1:500; 1:500 and 1:1000, or a range thereof. For example, in some embodiments, the ratio of Mg²⁺ to Ca²⁺ in the aqueous solution of divalent cations is between 1:1 and 1:10; 1:5 and 1:25; 1:10 and 1:50; 1:25 and 1:100; 1:50 and 1:500; or 1:100 and 1:1000.

The aqueous solution of divalent cations may comprise divalent cations derived from freshwater, brackish water, seawater, or brine (e.g., naturally occurring brines or anthropogenic brines such as geothermal plant wastewaters, desalination plant waste waters), as well as other salines having a salinity that is greater than that of freshwater, any of which may be naturally occurring or anthropogenic. Brackish water is water that is saltier than freshwater, but not as salty as seawater. Brackish water has a salinity ranging from about 0.5 to about 35 ppt (parts per thousand). Seawater is water from a sea, an ocean, or any other saline body of water that has a salinity ranging from about 35 to about 50 ppt. Brine is water saturated or nearly saturated with salt. Brine has a salinity that is about 50 ppt or greater. In some embodiments, the water source from which divalent cations are derived is a mineral rich (e.g., calcium-rich and/or magnesium-rich) freshwater source. In some embodiments, the water source from which divalent cations are derived is a naturally occurring saltwater source selected from a sea, an ocean, a lake, a swamp, an estuary, a lagoon, a surface brine, a subsurface brine, a deep brine, an alkaline lake, an inland sea, or the like. In some embodiments, the water source from which divalent cations are derived is a surface brine. In some embodiments, the water source from which divalent cations are derived is a subsurface brine. In some embodiments, the water source from which divalent cations are derived is a deep brine. In some embodiments, the water source from which divalent cations are derived is a Ca—Mg—Na—(K)—Cl; Na—(Ca)—SO₄—Cl; Mg—Na—(Ca)—SO₄—Cl; Na—CO₃—Cl; or Na—CO₃—SO₄—Cl brine. In some embodiments, the water source from which divalent cation are derived is an anthropogenic brine selected from a geothermal plant wastewater or a desalination wastewater.

In some embodiments, the brine is a subterranean brine which may be a convenient source for divalent cations, monovalent cations, proton-removing agents, or any combination thereof. Subterranean brines include naturally occurring or anthropogenic subterranean brines (e.g., an anthropogenic brine that has been injected into a subterranean site), many of which comprise concentrated aqueous saline compositions. The geological location of the subterranean brine may be below ground (e.g., subterranean site), optionally just below the Earth's surface, or even under water bodies such as Earth's oceans or lakes. A concentrated aqueous saline composition includes an aqueous solution which has a salinity of 10,000 ppm total dissolved solids (TDS) or greater, such as 20,000 ppm TDS or greater and including 50,000 ppm TDS or greater. A subterranean geological location includes a geological location that is located below ground level, such as a solid-fluid interface of the Earth's surface, such as a solid-gas interface as found on dry land where dry land meets the Earth's atmosphere, as well as a liquid-solid interface as found beneath a body of surface water (e.g., lack, ocean, stream, etc) where solid ground meets the body of water (where examples of this interface include lake beds, ocean floors, etc). For example, the subterranean location can be a location beneath land or a location beneath a body of water (e.g., oceanic ridge). For example, a subterranean location may be a deep geological alkaline aquifer or an underground well located in the sedimentary basins of a petroleum field, a subterranean metal ore, a geothermal field, or an oceanic ridge, among other underground locations. Such brines have been described in U.S. Provisional Application No. 61/371,620, filed 6 Aug. 2010, titled, “Calcium carbonate compositions and methods thereof,” which is incorporated herein by reference in its entirety.

Freshwater is often a convenient source of divalent cations (e.g., cations of alkaline earth metals such as, but not limited to, Ca²⁺ and Mg²⁺). Any number of suitable freshwater sources may be used, including freshwater sources ranging from sources relatively free of minerals to sources relatively rich in minerals. Mineral-rich freshwater sources may be naturally occurring, including any number of hard water sources, lakes, or inland seas. Some mineral-rich freshwater sources such as alkaline lakes or inland seas (e.g., Lake Van in Turkey) also provide a source of pH-modifying agents. Mineral-rich freshwater sources may also be anthropogenic. For example, a mineral-poor (soft) water may be contacted with a source of divalent cations such as alkaline earth metal cations (e.g., Ca²⁺, Mg²⁺, etc.) to produce a mineral-rich water that is suitable for methods and systems described herein. Divalent cations or precursors thereof (e.g. salts, minerals) may be added to freshwater (or any other type of water described herein) using any convenient protocol (e.g., addition of solids, suspensions, or solutions). In some embodiments, divalent cations selected from Ca²⁺ and Mg²⁺ are added to freshwater. In some embodiments, monovalent cations selected from Na⁺ and K⁺ are added to freshwater. In some embodiments, freshwater comprising Ca²⁺ is combined with magnesium silicates (e.g., olivine, serpentine, etc.), or products or processed forms thereof, yielding a solution comprising calcium and magnesium cations. In some embodiments, metal silicates (e.g., olivine, serpentine, wollastonite) are added to a solution that has become acidic due to carbonic acid formed from dissolution of carbon dioxide, which acidic solution dissolves the added metal silicate leading to the formation of Mg²⁺, magnesium compounds, Ca²⁺, calcium compounds, or mixtures thereof. In some embodiments, freshwater comprising Ca²⁺ is combined with combustion ash (e.g., fly ash, bottom ash, boiler slag), or products or processed forms thereof, yielding a solution comprising calcium and magnesium cations.

In some embodiments, an aqueous solution of divalent cations may be obtained from an industrial plant that is also providing a combustion gas stream. For example, in water-cooled industrial plants, such as seawater-cooled industrial plants, water that has been used by an industrial plant for cooling may then be used as water for producing precipitation material. If desired, the water may be cooled prior to entering a precipitation system of the invention. Such approaches may be employed, for example, with once-through cooling systems. For example, a city or agricultural water supply may be employed as a once-through cooling system for an industrial plant. Water from the industrial plant may then be employed for producing precipitation material, wherein output water has a reduced hardness and greater purity.

### Proton-Removing Agents

In some embodiments, the methods and systems of the invention include using a proton-removing agent. As used herein, a “proton-removing agent” that possesses sufficient basicity to remove one or more protons from a proton-containing species such as, but not limited to, carbonic acid, bicarbonate, hydronium, etc. A solution of proton-removing agents may be described in terms of alkalinity or the ability of the solution of proton-removing agents to neutralize acidic species to the equivalence point. In some embodiments, the proton-removing agent may be contacted with the gaseous stream of CO₂ before contacting the gaseous stream, with the catalyst. In some embodiments, the proton-removing agent may be contacted with the gaseous stream of CO₂ after contacting the gaseous stream with the catalyst. In some embodiments, the proton-removing agent may be contacted with the gaseous stream of CO₂ while contacting the CO₂ with the catalyst. In some embodiments, the proton-removing agent may be contacted with the gaseous stream of CO₂ and/or the aqueous solution including the hydrated CO₂ along with the source of divalent cations. It is to be understood that the order of the contact of the proton-removing agent and the catalyst with the source of CO₂ may vary depending on the required pH for the dissolution of the CO₂. In some embodiments, the use of the proton-removing agent is optional and the catalyst may be sufficient to form the hydrated CO₂ species.

In some embodiments, methods of the invention include contacting a volume of an aqueous solution of divalent cations with a source of CO₂ (to dissolve CO₂) and/or the aqueous solution including the hydrated CO₂ and subjecting the resultant solution to conditions that facilitate precipitation. In some embodiments, a volume of an aqueous solution of divalent cations is contacted with a source of CO₂ (to dissolve CO₂) and/or the aqueous solution including the hydrated CO₂ while subjecting the aqueous solution to conditions that facilitate precipitation.

The dissolution of CO₂ into the aqueous solution of divalent cations and/or the catalyst with optional proton-removing agent may produce carbonic acid, a species that may be in equilibrium with bicarbonate and/or carbonate. The catalyst may facilitate hydration of CO₂ to form an aqueous solution of hydrated CO₂ including bicarbonate and/or carbonate. The aqueous solution of hydrated CO₂ may then be treated with the solution of divalent cations, optionally including the proton-removing agent, to form the precipitation material and the composition of the invention. In order to produce carbonate- and/or bicarbonate-containing precipitation material, protons may be removed from various species (e.g. carbonic acid, bicarbonate, hydronium, etc.) in the divalent cation-containing solution to shift the equilibrium toward carbonate. As protons are removed, more CO₂ goes into solution.

In some embodiments, proton-removing agents and/or methods are used while contacting a divalent cation-containing aqueous solution with CO₂ and/or the aqueous solution including the hydrated CO₂ to increase CO₂ absorption in one phase of the precipitation reaction, wherein the pH may remain constant, increase, or even decrease, followed by a rapid removal of protons (e.g., by addition of a base) to cause rapid precipitation of carbonate- and/or bicarbonate-containing precipitation material. Protons may be removed from the various species (e.g. carbonic acid, bicarbonate, hydronium, etc.) by any convenient approach, including, but not limited to use of naturally occurring proton-removing agents, use of microorganisms and fungi, use of synthetic chemical proton-removing agents, recovery of man-made waste streams, and using electrochemical means.

Naturally occurring proton-removing agents encompass any proton-removing agents that can be found in the wider environment that may create or have a basic local environment. Some embodiments provide for naturally occurring proton-removing agents including minerals that create basic environments upon addition to solution. Such minerals include, but are not limited to, lime (CaO); periclase (MgO); iron hydroxide minerals (e.g., goethite and limonite); and volcanic ash. Methods for digestion of such minerals and rocks comprising such minerals are provided herein. Some embodiments provide for using naturally occurring bodies of water as a source of proton-removing agents, which bodies of water comprise carbonate, borate, sulfate, or nitrate alkalinity, or some combination thereof. Any alkaline brine (e.g., surface brine, subsurface brine, a deep brine, a subterranean brine, etc.) is suitable for use in the invention. In some embodiments, a surface brine comprising carbonate alkalinity provides a source of proton-removing agents. In some embodiments, a surface brine comprising borate alkalinity provides a source of proton-removing agents. In some embodiments, a subsurface brine comprising carbonate alkalinity provides a source of proton-removing agents. In some embodiments, a subsurface brine comprising borate alkalinity provides a source of proton-removing agents. In some embodiments, a deep brine comprising carbonate alkalinity provides a source of proton-removing agents. In some embodiments, a deep brine comprising borate alkalinity provides a source of proton-removing agents. Examples of naturally alkaline bodies of water include, but are not limited to surface water sources (e.g. alkaline lakes such as Mono Lake in California) and ground water sources (e.g. basic aquifers such as the deep geologic alkaline aquifers located at Searles Lake in California). Other embodiments provide for use of deposits from dried alkaline bodies of water such as the crust along Lake Natron in Africa's Great Rift Valley.

In some embodiments, organisms that excrete basic molecules or solutions in their normal metabolism are used as proton-removing agents. Examples of such organisms are fungi that produce alkaline protease (e.g., the deep-sea fungus Aspergillus ustus with an optimal pH of 9) and bacteria that create alkaline molecules (e.g., cyanobacteria such as Lyngbya sp. from the Atlin wetland in British Columbia, which increases pH from a byproduct of photosynthesis). In some embodiments, organisms are used to produce proton-removing agents, wherein the organisms (e.g., Bacillus pasteurii, which hydrolyzes urea to ammonia) metabolize a contaminant (e.g. urea) to produce proton-removing agents or solutions comprising proton-removing agents (e.g., ammonia, ammonium hydroxide). In some embodiments, organisms are cultured separately from the precipitation reaction mixture, wherein proton-removing agents or solution comprising proton-removing agents are used for addition to the precipitation reaction mixture. In some embodiments, naturally occurring or manufactured enzymes are used in combination with proton-removing agents to invoke precipitation of precipitation material. Carbonic anhydrase, which is an enzyme produced by plants and animals, accelerates transformation of carbonic acid to bicarbonate in aqueous solution. As such, carbonic anhydrase may be used to enhance dissolution of CO₂ and accelerate precipitation of precipitation material, as described in further detail herein.

Chemical agents for effecting proton removal generally refer to synthetic chemical agents that are produced in large quantities and are commercially available. For example, chemical agents for removing protons include, but are not limited to, hydroxides, organic bases, super bases, oxides, ammonia, and carbonates. Hydroxides include chemical species that provide hydroxide anions in solution, including, for example, sodium hydroxide (NaOH), potassium hydroxide (KOH), calcium hydroxide (Ca(OH)₂), or magnesium hydroxide (Mg(OH)₂). Organic bases are carbon-containing molecules that are generally nitrogenous bases including primary amines such as methyl amine, secondary amines such as diisopropylamine, tertiary such as diisopropylethylamine, aromatic amines such as aniline, heteroaromatics such as pyridine, imidazole, and benzimidazole, and various forms thereof. In some embodiments, an organic base selected from pyridine, methylamine, imidazole, benzimidazole, histidine, and a phosphazene is used to remove protons from various species (e.g., carbonic acid, bicarbonate, hydronium, etc.) for precipitation of precipitation material. In some embodiments, ammonia is used to raise pH to a level sufficient to precipitate precipitation material from a solution of divalent cations and an industrial waste stream. Super bases suitable for use as proton-removing agents include sodium ethoxide, sodium amide (NaNH₂), sodium hydride (NaH), butyl lithium, lithium diisopropylamide, lithium diethylamide, and lithium bis(trimethylsilyl)amide. Oxides including, for example, calcium oxide (CaO), magnesium oxide (MgO), strontium oxide (SrO), beryllium oxide (BeO), and barium oxide (BaO) are also suitable proton-removing agents that may be used. Carbonates for use in the invention include, but are not limited to, sodium carbonate.

In addition to comprising cations of interest and other suitable metal forms, waste streams from various industrial processes may provide proton-removing agents. Such waste streams include, but are not limited to, mining wastes; fossil fuel burning ash (e.g., combustion ash such as fly ash, bottom ash, boiler slag); slag (e.g. iron slag, phosphorous slag); cement kiln waste; oil refinery/petrochemical refinery waste (e.g. oil field and methane seam brines); coal seam wastes (e.g. gas production brines and coal seam brine); paper processing waste; water softening waste brine (e.g., ion exchange effluent); silicon processing wastes; agricultural waste; metal finishing waste; high pH textile waste; and caustic sludge. Mining wastes include any wastes from the extraction of metal or another precious or useful mineral from the earth. In some embodiments, wastes from mining are used to modify pH, wherein the waste is selected from red mud from the Bayer aluminum extraction process; waste from magnesium extraction from seawater (e.g., Mg(OH)₂ such as that found in Moss Landing, Calif.); and wastes from mining processes involving leaching. For example, red mud may be used to modify pH as described in U.S. Provisional Patent Application No. 61/161,369, filed 18 Mar. 2009, which is incorporated herein by reference in its entirety. Fossil fuel burning ash, cement kiln dust, and slag, collectively waste sources of metal oxides, further described in U.S. patent application Ser. No. 12/486,692, filed 17 Jun. 2009, the disclosure of which is incorporated herein in its entirety, may be used in alone or in combination with other proton-removing agents to provide proton-removing agents for the invention. Agricultural waste, either through animal waste or excessive fertilizer use, may contain potassium hydroxide (KOH) or ammonia (NH₃) or both. As such, agricultural waste may be used in some embodiments of the invention as a proton-removing agent. This agricultural waste is often collected in ponds, but it may also percolate down into aquifers, where it can be accessed and used.

Electrochemical methods are another means to remove protons from various species in a solution, either by removing protons from solute (e.g., deprotonation of carbonic acid or bicarbonate) or from solvent (e.g., deprotonation of hydronium or water). Deprotonation of solvent may result, for example, if proton production from CO₂ dissolution matches or exceeds electrochemical proton removal from solute molecules. In some embodiments, low-voltage electrochemical methods are used to remove protons, for example, as CO₂ is dissolved in the precipitation reaction mixture or a precursor solution to the precipitation reaction mixture (i.e., a solution that may or may not contain divalent cations). In some embodiments, CO₂ dissolved in an aqueous solution that does not contain divalent cations is treated by a low-voltage electrochemical method to remove protons from carbonic acid, bicarbonate, hydronium, or any species or combination thereof resulting from the dissolution of CO₂. A low-voltage electrochemical method operates at an average voltage of 2.8, 2.7, 2.6, 2.5, 2.4, 2.3, 2.2, 2.1, or 2.0 V or less, such as 1.9, 1.8, 1.7, 1.6 V or less, such as 1.5, 1.4, 1.3, 1.2, 1.1 V or less, such as 1.0 V or less, such as 0.9 V or less, 0.8 V or less, 0.7 V or less, 0.6 V or less, 0.5 V or less, 0.4 V or less, 0.3 V or less, 0.2 V or less, or 0.1 V or less. Low-voltage electrochemical methods that do not generate chlorine gas are convenient for use in systems and methods of the invention. Low-voltage electrochemical methods to remove protons that do not generate oxygen gas are also convenient for use in systems and methods of the invention. In some embodiments, low-voltage methods do not generate any gas (e.g., chlorine, oxygen, etc.) at the anode. In some embodiments, low-voltage electrochemical methods generate hydrogen gas at the cathode and transport it to the anode where the hydrogen gas is oxidized and converted to protons. In some embodiments, the systems of the invention include a delivery system, such as, for example, a duct, to transport the hydrogen gas generated at the cathode to the anode. In some embodiments, the anode is a gas diffusion anode. Electrochemical methods that do not generate hydrogen gas may also be convenient. In some instances, electrochemical methods to remove protons do not generate any gaseous by-byproduct. Electrochemical methods for effecting proton removal are further described in U.S. patent application Ser. No. 12/344,019, filed 24 Dec. 2008; U.S. patent application Ser. No. 12/375,632, filed 23 Dec. 2008; International Patent Application No. PCT/US08/088,242, filed 23 Dec. 2008; International Patent Application No. PCT/US09/32301, filed 28 Jan. 2009; and International Patent Application No. PCT/US09/48511, filed 24 Jun. 2009, each of which are incorporated herein by reference in their entirety.

Alternatively, electrochemical methods may be used to produce caustic molecules (e.g., hydroxide) through, for example, the chlor-alkali process, or a modification thereof, including low-voltage electrochemical methods such as that described above. In such low-voltage electrochemical methods for producing caustic molecules, carbon dioxide is not dissolved in electrolyte. In such low-voltage electrochemical methods for producing caustic molecules (e.g., NaOH), the applied voltage across the anode and cathode is 2.8, 2.7, 2.6, 2.5, 2.4, 2.3, 2.2, 2.1, or 2.0 V or less, such as 1.9, 1.8, 1.7, 1.6 V or less, such as 1.5, 1.4, 1.3, 1.2, 1.1 V or less, such as 1.0 V or less, such as 0.9 V or less, 0.8 V or less, 0.7 V or less, 0.6 V or less, 0.5 V or less, 0.4 V or less, 0.3 V or less, 0.2 V or less, or 0.1 V or less. Low-voltage electrochemical methods for producing caustic molecules (e.g., NaOH) that do not generate chlorine gas are convenient for use in systems and methods of the invention. Low-voltage electrochemical methods for producing caustic molecules (e.g., NaOH) that do not generate oxygen gas are also convenient for use in systems and methods of the invention. In some embodiments, low-voltage methods for producing caustic molecules (e.g., NaOH) that do not generate any gas (e.g., chlorine, oxygen, etc.) at the anode. In some embodiments, low-voltage electrochemical methods for producing caustic molecules (e.g., NaOH) generate hydrogen gas at the cathode and transport it to the anode where the hydrogen gas is oxidized and converted to protons. In some embodiments, electrochemical systems of the invention include a delivery system, such as, for example, a duct or pipe to transport the hydrogen gas generated at the cathode to the anode. Hydrogen generated in electrochemical systems using methods described herein may be harvested and used for other purposes (e.g., sold, combusted with oxygen to produce electricity and/or water for the process). Additional electrochemical approaches that may be used in systems and methods of the invention include, but are not limited to, those described in U.S. Provisional Patent Application No. 61/081,299, filed 16 Jul. 2008, and U.S. Provisional Patent Application No. 61/091,729, the disclosures of which are incorporated herein by reference. Combinations of the above mentioned sources of proton-removing agents and methods for effecting proton removal may be employed.

### Catalyst

A “catalyst” as used herein, includes any agent capable of reducing the activation energy for producing CO₂ hydration products or hydrated CO₂. The “hydrated CO₂” or “CO₂ hydration product” as used herein, includes any form of hydrated CO₂ including H₂CO₃ (aq), HCO₃⁻ (aq), and/or CO₃²⁻ (aq)). Such catalysts include, but are not limited to, inorganic catalysts, organic catalysts, and biocatalysts. Such catalysts include naturally occurring catalysts or synthetic catalysts.

In some embodiments, the carbonate in the composition comprising hydrated CO₂ may result in the partial or complete inactivation of the catalyst activity owing to product inhibition. In such embodiments, the aqueous solution including the hydrated CO₂ and the catalyst (if not immobilized) may be withdrawn from the solution and an aqueous solution of the divalent cations may be added to the withdrawn solution to precipitate the precipitation material. The supernatant from the precipitation material including the catalyst (if not immobilized) may be re-circulated back to the catalyst solution to further dissolve gaseous stream of CO₂. Such withdrawal of the aqueous solution including the hydrated CO₂ may prevent the partial or complete inactivation of the catalyst. For example, carbonic anhydrase may be partially or completely inactivated by the presence of carbonate and/or bicarbonate in the solution of hydrated CO₂. Such inactivation may be prevented by withdrawing the aqueous solution of the hydrated CO₂ and re-circulating the supernatant, as above.

Inorganic catalysts include catalysts that are not organic catalysts, which organic catalysts generally comprise compounds based on one or more units of carbon bonded to hydrogen (i.e., C—H units). Inorganic catalysts suitable for use with the invention include, but are not limited to, anions such as arsenate, hypochlorite, and hypobromite; cations such as Zn²⁺, Cd²⁺, SO₄²⁻, CO²⁺, Cu²⁺, Fe³⁺, and salts that may generate such anions and cations such as ZnCl₂, CdCl₂, KCl, and CaCl₂. It should be understood that an inorganic catalyst may include both inorganic as well as organic components. Some inorganic catalysts, for example, comprise a metal and organic ligands (e.g., organometallic compounds such as ferrocene, methylcyclopentadienyl manganese tricarbonyl, etc.). These catalysts are well within the scope of the invention. Indeed, many catalysts suitable for use in the invention are organometallic catalysts that structurally mimic the active site of enzymes such as carbonic anhydrase (below). For example, tris-(pyrazolyl)hydroborato and tris(imidazolyl)phosphine complexes of zinc are suitable for use in the invention.

Organic catalysts, as above, are catalysts that generally comprise compounds based on one or more C—H units; however, it should be understood that some compounds are considered to be organic but do not contain a C—H unit, non-limiting examples being oxalic acid and urea. Biocatalysts are biological systems capable of catalyzing chemical reactions (e.g., hydration of CO₂) such as microbial communities; whole organisms or cells; cell-free extracts; or purified or catalytic enzymes. Biocatalysts suitable for use with the invention include, but are not limited to, enzymes, antibodies, liposomes, microorganisms, animal cells, plant cells, and the like. Fractions of enzymes, complexes, or combinations thereof may also be used to lower the activation energy for reactions in which CO₂-derived species are produced in water. Fractions of enzymes may comprise, for example, specific sub-units of enzymes, such as catalytic sub-units. Fractions of microorganisms, animal cells, or plant cells may comprise, for example, specific sub-cellular organelles or compartments such as cellular membranes, ribosome's, mitochondria, chloroplasts, or fractions such as cytoplasmic or nuclear extracts.

Biocatalysts such as enzymes are capable of catalyzing the formation of bicarbonate from carbon dioxide and may be used in the invention. The enzyme carbonic anhydrase is one such biocatalyst that may be used in the invention. Carbonic anhydrase, unless noted otherwise, comprises any carbonic anhydrase of the five families of carbonic anhydrases (e.g., α, β, γ, δ and ε), any of which, whether purified from a natural source or obtained by processes involving recombinant DNA technology, are suitable for use in the invention. Modified forms of carbonic anhydrase (e.g., forms engineered for increased stability to high temperature, pH, etc.; forms engineered for better turnover) obtained by processes involving recombinant DNA technology are also suitable for use in the invention. Carbonic anhydrase further includes any combination of carbonic anhydrases (i.e., mixtures of different carbonic anhydrases), and further, any combination of carbonic anhydrase with a previously mentioned catalyst or additive, including carbonic anhydrase activators such as, but not limited to, L- and D-histidine, L- and D-phenylalanine, beta-Ala-His, histamine, trisubstituted pyridinium azole compounds, tetrasubstituted pyridinium azole compounds, and L-adrenaline. Without being bound by theory, activators of CA bind to the entrance of the active site (near His64) and increase k_{cat} for the hydration of CO₂ by enhancing the activity of the proton shuttle. Despite their common catalytic activity, carbonic anhydrases from different families do not have significant homology (i.e., sequence similarity); however, most carbonic anhydrases contain a zinc ion at the active site. At least one family has been reported to comprise a cadmium ion at the active site. Mechanistically, the available evidence to date suggests that members of the carbonic anhydrase family share a similar ping-pong mechanism. Without being bound by theory, the active site of carbonic anhydrase contains a Zn^{II} ion with a bound hydroxyl group (Zn^{II}-OH) surrounded by three histidine residues held in a distorted tetrahedral geometry. Evidence suggests that the Zn^{II}-bound hydroxyl group attacks CO₂ to initiate hydrolysis of a weakly bound CO₂ to produce bicarbonate, which is subsequently displaced from the Zn^{II} ion by a molecule of water. The Zn^{II}-bound water loses a proton to His64, which acts as a proton shuttle, to generate a new Zn^{II}-OH for another round of catalysis. It is generally accepted that this proton is shuttled to buffers in solution by a series of intramolecular and intermolecular proton-transfer steps. Perhaps unexpectedly, the transfer of a proton from the Zn^{II}-bound water to buffer molecules appears to be the rate-limiting step in catalysis. For additional mechanistic details, see Krishnamurthy et al. Chemical Reviews, 2008, 108, 3, 946-1051.)

Catalysts may be free, immobilized, or some combination thereof, in a processor of the invention. As such, catalysts may be in a component of the processor such as, but not limited to, a gas-liquid contactor or a gas-liquid-solid contactor. Because catalysts may be expensive, retention of catalysts in the processor may be desired. As such, embodiments of the invention provide for immobilization of catalysts. Immobilization of catalysts may be effected by immobilization on an immobilization material, which material may serve to both immobilize and stabilize catalysts of the invention. Furthermore, the immobilization material may interfere as little as possible with the catalyzed reaction. For example, immobilization material, onto which an enzyme may be immobilized, may be permeable to compounds smaller than the immobilized enzyme such that the desired reaction (e.g., catalytic conversion of carbon dioxide to bicarbonate) may be catalyzed by the immobilized enzyme.

Typically, a preparation of free enzyme in solution (e.g., carbonic anhydrase in a divalent cation-containing solution) may lose its specific activity within a few hours to a few days, whereas a preparation of enzyme immobilized on an immobilization material may retain its specific activity for 5 days to 1500 days, or 5 days to 1000 days, or 5 days to 500 days, or 5 days to 250 days, or 5 days to 100 days, or 5 days to 50, or 25 days to 1500 days, or 25 days to 1000 days, or 25 days to 500 days, or 25 days to 250 days, or 25 days to 100 days, or 25 days to 50, or 50 days to 1500 days, or 50 days to 1000 days, or 50 days to 500 days, or 50 days to 250 days, or 50 days to 100 days, or 100 days to 1500 days, or 100 days to 1000 days, or 100 days to 500 days, or 100 days to 250 days, or 250 days to 1500 days, or 250 days to 1000 days, or 250 days to 500 days, or 500 days to 1500 days, or 500 days to 1000 days, or 1000 days to 1500 days. In some embodiments, a preparation of immobilized enzyme may retain at least 75% or between 10-95% of its initial specific activity for at least 5 days, 10 days, 25 days, 50 days, 100 days, 250 days, 500 days, 1000 days, 1500 days, or more, or 5 days to 1500 days, or 5 days to 1000 days, or 5 days to 500 days, or 5 days to 250 days, or 5 days to 100 days, or 5 days to 50, or 25 days to 1500 days, or 25 days to 1000 days, or 25 days to 500 days, or 25 days to 250 days, or 25 days to 100 days, or 25 days to 50, or 50 days to 1500 days, or 50 days to 1000 days, or 50 days to 500 days, or 50 days to 250 days, or 50 days to 100 days, or 100 days to 1500 days, or 100 days to 1000 days, or 100 days to 500 days, or 100 days to 250 days, or 250 days to 1500 days, or 250 days to 1000 days, or 250 days to 500 days, or 500 days to 1500 days, or 500 days to 1000 days, or 1000 days to 1500 days.

In various embodiments, a preparation of immobilized enzyme may retain at least about 75%, 80%, 85%, 90%, 95%, more than 95%, or between 10-95%, or between 10-75%, or between 10-50%, or between 10-25%, or between 25-95%, or between 25-50%, or between 50-95%, or between 50-75%; or between 75-95%, of its initial specific activity for at least 5 days, 10 days, 25 days, 50 days, 100 days, 250 days, 500 days, 1000 days, 1500 days, or more, or 5 days to 1500 days, or 5 days to 1000 days, or 5 days to 500 days, or 5 days to 250 days, or 5 days to 100 days, or 5 days to 50, or 25 days to 1500 days, or 25 days to 1000 days, or 25 days to 500 days, or 25 days to 250 days, or 25 days to 100 days, or 25 days to 50, or 50 days to 1500 days, or 50 days to 1000 days, or 50 days to 500 days, or 50 days to 250 days, or 50 days to 100 days, or 100 days to 1500 days, or 100 days to 1000 days, or 100 days to 500 days, or 100 days to 250 days, or 250 days to 1500 days, or 250 days to 1000 days, or 250 days to 500 days, or 500 days to 1500 days, or 500 days to 1000 days, or 1000 days to 1500 days. Thus, immobilization of an enzyme such as carbonic anhydrase, besides allowing for retention of the actual enzyme, may provide a significant advantage in stability and/or activity. With respect to stabilizing the enzyme, the immobilization material may provide a chemical and mechanical barrier to impede or prevent enzyme denaturation. The immobilization material may, for example, physically confine the enzyme, preventing the enzyme from unfolding from its three-dimensional structure, which is one mechanism of enzyme denaturation.

Enzyme activity may be measured by analytical methods comprising chemiluminescence, electrochemistry, UV-Vis spectroscopy, radiochemistry, fluorescence, or the like. For example, fluorescence may be used to measure enzyme activity. In some embodiments, a preparation of immobilized enzyme may retain at least 75% or between 10-95% of its initial specific activity while continuously catalyzing a chemical transformation, wherein the activity is measured using an analytical method comprising chemiluminescence, electrochemistry, UV-Vis spectroscopy, radiochemistry, or fluorescence. A properly immobilized enzyme (i.e., a preparation of immobilized enzyme that retains significant specific activity) may be physically confined in a certain region of the immobilization material. There are a variety of methods for immobilization, including, but not limited to, carrier-binding (e.g., physical adsorption, ionic binding, covalent binding), cross-linking, and entrapping. Carrier-binding may be used with enzymes of the invention (e.g., carbonic anhydrase) to bind the enzymes to water-insoluble carriers. In some embodiments, cross-linking may be used to intermolecularly cross-link enzymes using bi-functional or multi-functional reagents. In some embodiments, entrapping may be used to incorporate enzymes into semi-permeable material or lattices thereof. The method by which an enzyme of the invention is immobilized may not be critical provided the enzyme is immobilized and stabilized. In addition, the preparation of immobilized enzyme may retain a significant portion of its specific activity. As such, the immobilization material may be permeable to compounds smaller than the enzyme such that compounds on which the enzyme acts are provided to the enzyme. For example, the immobilization material may be permeable to carbon dioxide and bicarbonate and/or carbonate when the immobilized enzyme is carbonic anhydrase. The immobilization material may also be permeable to compounds and agents that facilitate reactions catalyzed by immobilized enzymes. For example, the immobilization material may be permeable to water and various proton-removing agents that facilitate the carbonic anhydrase-catalyzed production of bicarbonate and/or carbonate from carbon dioxide.

The immobilization material may be prepared in a manner such that it contains internal pores, channels, openings, or some combination thereof, which simultaneously facilitate the movement of compounds through the immobilization material and constrain the enzyme to substantially the same space within the immobilization material. For example, an enzyme (e.g., carbonic anhydrase) may be located within a pore of the immobilization material and a compound (e.g., carbon dioxide or a solvated or hydrated form thereof) may travel in and out of the immobilization material through channels (e.g., interconnected pores). A properly immobilized enzyme may be confined to a space that is substantially the same size and shape as the enzyme, and the preparation of such properly immobilized enzyme may retain a significant portion of its specific activity. Such constraint, while allowing for retention of catalytic activity, may further inhibit denaturation (e.g., unfolding) of the enzyme. In some embodiments, the immobilization material pores, channels, openings, or some combination thereof have physical dimensions that may satisfy the above requirements and that depend upon the size and shape of the specific enzyme (e.g., carbonic anhydrase) to be immobilized.

Immobilization material for use with the invention may be in a form including, but not limited to, beads (e.g., silica beads); fabrics; fibers (e.g., graphite fibers); gel matrices; membranes (e.g., cellulose membranes); particulates; porous surfaces; rods (e.g., carbon rods); and tubes (e.g., carbon tubes). Immobilization material suitable for use in the invention may include, but not limited to, alumina; bentonite; biopolymers such as cellulose, starch, proteins (e.g., albumin), and peptides; calcium carbonate; calcium phosphate (e.g., calcium phosphate gel); carbon; ceramic supports; clay; porous metal structures; collagen; glass; hydroxyapatite; ion-exchange resins; kaolin; polymer meshes (e.g., nylon); polysaccharides (e.g., polysaccharides surfaces or gels); phenolic polymers; polyaminostyrene; polyacrylamide (e.g., a polyacrylamide gel); poly(acryloyl morpholine) (e.g., a poly(acryloyl morpholine) gel); polypropylene; polymer hydrogels; sephadex; sepharose; treated silicon oxides; silica gel; Teflon®-brand PTFE; and the like.

The catalyst may be immobilized in or on immobilization material using physical or chemical methods, wherein the methods include, but not limited to, physical attraction, adsorption, ionic bonding, covalent bonding (e.g., coordinate covalent bonding such as chelation), or other methods for immobilizing or entrapping catalyst. Furthermore, the catalyst (e.g., enzyme) may be used in its native form, or it may be cross-linked or co-cross-linked with other chemicals to enhance its activity.

The catalyst may be entrapped in a gel or polymer matrix, stabilized in a micellar structure, and/or incorporated into the substance of the matrix itself. In some embodiments, the biocatalysts may also be entrapped in a porous substrate, for example, an insoluble gel particle such as silica, alginate, alginate/chitosan, alginate/carboxymethylcellulose, etc. For example, an aqueous solution of an enzyme may be mixed with chitosan and a polyfunctional cross-linking agent (e.g., alginate) to form a gel, which may then be treated with a reducing agent to produce a granular material comprising the active enzyme. In some embodiments, biocatalysts may also be immobilized on solid packing in suspension in the liquid, such as enzymes covalently bound to plastic packing. In some embodiments, enzymes may be in a free state, or chemically linked in an albumin or PEG network. In some embodiments, the enzyme may be wholly or partially encapsulated in a suitable material such as cellulose nitrate capsules, polyvinyl alcohol capsules, starch capsules, or liposome preparations.

The catalyst (e.g., an enzyme such as carbonic anhydrase) may be immobilized on a membrane such as a membrane having selective permeability. Selectivity of the membrane may be based on, for example, size, charge, or some other characteristic. Permeable membranes, both natural and artificial, may be used in the invention, including, but not limited to, lipid bilayers such as black lipid membranes, supported lipid bilayers, and semi-permeable plastic membranes. In some embodiments, the membrane with selective permeability serves to maintain separation of the catalyst (e.g., an enzyme) from products of the invention. For example, the membrane with selective permeability may serve to separate carbonic anhydrase from a buildup of bicarbonate and/or carbonate (which may inhibit carbonic anhydrase).

In some embodiments, the immobilization material may have a micellar or inverted micellar structure comprising amphipathic molecules. Molecules making up a micelle are generally amphipathic, which include, but not limited to, both polar, hydrophilic groups and non-polar, hydrophobic groups. Amphipathic molecules may aggregate to form a micellar structure, where the polar groups are on the surface of the structure and the hydrocarbon, non-polar groups are inside the micellar structure. Inverted micelles have the opposite orientation of polar groups and non-polar groups. The amphipathic molecules making up micellar structures may be arranged in a variety of ways so long as the polar groups are in proximity to each other and the non-polar groups are in proximity to each other. Also, the amphipathic molecules may form a bilayer with the non-polar groups pointing toward each other and the polar groups pointing away from each other. In some embodiments, a bilayer may form in which polar groups point toward each other in the bilayer, while non-polar groups point away from each other.

In some embodiments, an enzyme preparation comprising enzyme immobilized in or on an immobilization material may result in retention of at least 50%, 60%, 70%, 80%, 90%, 95%, 97%, 98%, 99%, or 99.9% of the initial specific activity for at least 5 days when continuously catalyzing a certain chemical transformation (e.g., carbon dioxide to bicarbonate). In some embodiments, the enzyme preparation may result in retention of at least 50%, 60%, 70%, 80%, 90%, 95%, 97%, 98%, 99%, or 99.9% of the initial specific activity for at least 30 days when continuously catalyzing a chemical transformation. In some embodiments, the enzyme preparation may result in retention of at least 50%, 60%, 70%, 80%, 90%, 95%, 97%, 98%, 99%, or 99.9% of the initial specific activity for at least 60 days when continuously catalyzing a chemical transformation. In some embodiments, the enzyme preparation may result in retention of at least 50%, 60%, 70%, 80%, 90%, 95%, 97%, 98%, 99%, or 99.9% of the initial specific activity for at least 90 days when continuously catalyzing a chemical transformation. In some embodiments, the enzyme preparation may result in retention of at least 50%, 60%, 70%, 80%, 90%, 95%, 97%, 98%, 99%, or 99.9%, or between 50-95% of the initial specific activity for between 5-90 days when continuously catalyzing a chemical transformation.

In some embodiments, an enzyme preparation comprising enzyme immobilized in or on an immobilization material acting at 12° C. for at least 18 hours may result in retention of at least 50%, 60%, 70%, 80%, 90%, 95%, 97%, 98%, 99%, or 99.9% of the specific activity of an otherwise identical preparation comprising free enzyme acting at room temperature for the same amount of time. In some embodiments, the enzyme preparation acting at a temperature of at least 21° C. for at least 18 hours may result in retention of at least 50%, 60%, 70%, 80%, 90%, 95%, 97%, 98%, 99%, or 99.9% of the specific activity of an otherwise identical preparation of free enzyme acting at room temperature for the same amount of time. In some embodiments, the enzyme preparation acting at a temperature of at least 35° C. for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation of free enzyme acting at room temperature for the same amount of time. In some embodiments, the enzyme preparation acting at a temperature of at least 65° C. for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation of free enzyme acting at room temperature for the same amount of time. In some embodiments, the enzyme preparation acting at a temperature of at least 95° C. for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation of free enzyme acting at room temperature for the same amount of time. In some embodiments, the enzyme preparation acting at a temperature of between 12-95° C. for between 18-60 hours may result in retention of at least 1%, 5%, 10%, 20%, 30%, 40%, 50%, 60%, 70%, 80%, 90%, 95%, 97%, 98%, 99%, or 99.9%, or between 1-95% of the specific activity of an otherwise identical preparation of free enzyme acting at room temperature for the same amount of time.

An enzyme preparation comprising enzyme immobilized in or on an immobilization material acting at a pH of at least pH 7 for at least 18 hours may result in retention of at least 50%, 60%, 70%, 80%, 90%, 95%, 97%, 98%, 99%, or 99.9% of the specific activity of an otherwise identical preparation comprising free enzyme acting at an optimal pH for the same amount of time. In some embodiments, the enzyme preparation acting at a pH of at least pH 8 for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation comprising free enzyme acting at an optimal pH for the same amount of time. In some embodiments, the enzyme preparation acting at a pH of at least pH 9 for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation comprising free enzyme acting at an optimal pH for the same amount of time. In some embodiments, the enzyme preparation acting at a pH of at least pH 10 for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation comprising free enzyme acting at an optimal pH for the same amount of time. In some embodiments, the enzyme preparation acting at a pH of at least pH 11 for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation comprising free enzyme acting at an optimal pH for the same amount of time. In some embodiments, the enzyme preparation acting at a pH of at least pH 12 for at least 18 hours may result in retention of at least 1%, 5%, 10%, 20%, 30%, 40%, 50%, 60%, 70%, 80%, 90%, 95%, 97%, 98%, 99%, or 99.9% of the specific activity of an otherwise identical preparation comprising free enzyme acting at an optimal pH for the same amount of time. In some embodiments, the enzyme preparation acting at a pH of between pH 7-12 for at least 18 hours may result in retention of at least 1%, 5%, 10%, 20%, 30%, 40%, 50%, 60%, 70%, 80%, 90%, 95%, 97%, 98%, 99%, or 99.9%, or between 1-95% of the specific activity of an otherwise identical preparation comprising free enzyme acting at an optimal pH for the same amount of time.

In some embodiments, the immobilized enzymes such as carbonic anhydrase acting at a pH of less than pH 2 for at least 1 hour may result in retention of at least 1%, 5%, 10%, 20%, 30%, 40%, 50%, 60%, 70%, 80%, 90%, 95%, 97%, 98%, 99%, or 99.9% of the specific activity of an otherwise identical free enzyme acting at an optimal pH for the same amount of time. In some embodiments, the immobilized enzymes such as carbonic anhydrase acting at a pH of between 0.5-2 for at least 1 hour may result in retention of at least 1%, 5%, 10%, 20%, 30%, 40%, 50%, 60%, 70%, 80%, 90%, 95%, 97%, 98%, 99%, or 99.9%, or between 1-95% of the specific activity of an otherwise identical free enzyme acting at an optimal pH for the same amount of time.

### Methods

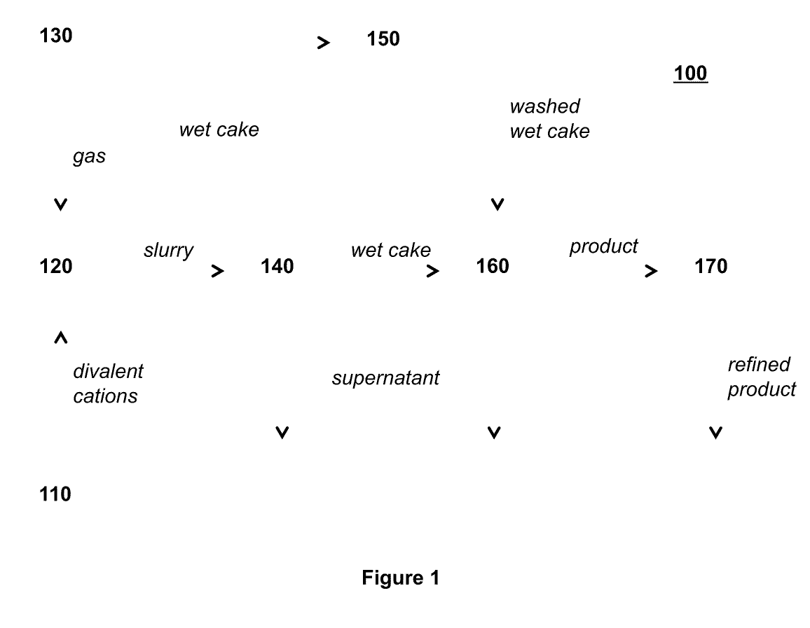

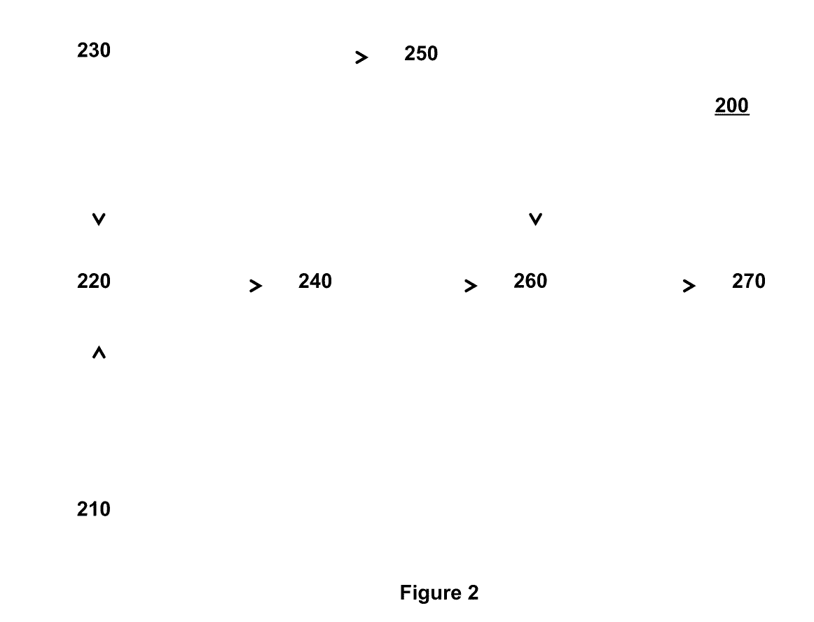

In some embodiments, the invention provides a method for processing a CO₂-containing gas with a catalyst and producing a storage-stable precipitation material including carbonates and/or bicarbonates. The precipitation material results in the compositions of the invention. In some embodiments, the CO₂-containing gas may be employed in a precipitation process to produce a precipitation material comprising carbonates and/or bicarbonates. FIG. 1 provides a schematic flow diagram of a method for processing a CO₂-containing gas with a catalyst that may be implemented in a system, wherein the system (e.g., system 200 of FIG. 2) may be a stand-alone plant or an integrated part of another type of plant (e.g., a power generation plant, a cement production plant, etc.). In some embodiments, methods include contacting a source of CO₂ 130 with a catalyst in a processing step 120. The processing step may additionally include a proton-removing agent. The amount of proton-removing agent added to the catalyst or the source of divalent cations may vary depending on the desired pH of the solution. The CO₂, after coming in contact with the catalyst and optionally the proton-removing agent, may result in the aqueous solution including hydrated CO₂ such as bicarbonate and/or carbonate ions. In some embodiments, the source of divalent cations may be contacted with the solution containing the catalyst and optionally the proton-removing agent before the catalyst is contacted with the source of CO₂. In some embodiments, the source of divalent cations may be contacted with the solution containing the catalyst and optionally the proton-removing agent after the catalyst has been contacted with the source of CO₂ and has resulted in the aqueous solution including hydrated CO₂. In some embodiments, the source of divalent cations may be contacted with the solution containing the catalyst and optionally the proton-removing agent while the catalyst is being contacted with the source of CO₂. As shown in FIG. 1, a divalent cation-containing solution may be sourced in step 110 and delivered to a processor to be processed (e.g., subjected to conditions suitable for precipitation of precipitation material) in a processing step (120), wherein the solution is delivered to the processor via a pipeline or another convenient apparatus. Also shown in FIG. 1, a CO₂-containing gas may be sourced in step 130 and delivered to the processor to be processed. In some embodiments, the aqueous solution including hydrated CO₂ such as bicarbonate and/or carbonate ions, is removed from the catalyst and is then contacted with the source of divalent cations. Methods for producing the precipitation material or storage stable material include sourcing divalent cations, CO₂-containing gas, proton-removing agents, and catalysts followed by processing the CO₂-containing gas with the catalyst to produce the storage stable material. It is to be understood that FIG. 1 is for illustration purposes only and in no way limits the scope of the invention.

In some embodiments, the processing step includes the treatment of the gaseous stream of CO₂ with the catalyst optionally in the presence of the proton-removing agent to result in the aqueous solution of the hydrated CO₂, which may then be withdrawn from the processor and treated with the aqueous solution of the divalent cations outside of the processor to form the precipitation material. The supernatant obtained from the precipitated material, optionally containing the catalyst (if not immobilized), is then re-circulated back to the processor for dissolving the CO₂.

Catalysts of the invention enhance CO₂ absorption by providing a reaction-based sink for dissolved CO₂ at the liquid side boundary layer. This may have the effect of steepening the concentration gradient, thereby increasing the flux of CO₂ across the gas-liquid interface.

A solution, optionally containing divalent cations (e.g., alkaline earth metal ions such as Ca²⁺ and Mg²⁺) may first be contacted with a CO₂-containing gas, wherein the solution may further comprise a catalyst, which lowers the activation energy for producing CO₂ hydration products (i.e., any conjugate acids or bases resulting from addition of water to CO₂, including, for example, H₂CO₃ (aq), HCO₃⁻ (aq), and CO₃²⁻ (aq)) from carbon dioxide relative to analogous uncatalyzed reactions for producing the same species. In some embodiments, it may be desirable to catalyze formation of bicarbonate from carbon dioxide, for example, with a biocatalyst such as an enzyme (e.g., carbonic anhydrase). Without catalysis, hydration of carbon dioxide to bicarbonate undergoes an intermediate hydration reaction, wherein the process may be described by the following reaction.

CO₂(aq)⇄H₂CO₃(aq)⇄H⁺⁽aq)+HCO₃⁻(aq)(without catalyst)

With a catalyst, the hydration of carbon dioxide to bicarbonate need not proceed by means of an intermediate hydration. As shown in the equation below, the catalyst may act on dissolved CO₂ directly.

CO₂(aq)⇄H⁺(aq)+HCO₃⁻(aq)(with catalyst)

Any agent capable of reducing the activation energy for producing CO₂ hydration products (e.g., H₂CO₃ (aq), HCO₃⁻(aq), CO₃²⁻(aq)) is suitable for the invention, including, but not limited to, inorganic catalysts, organic catalysts, and biocatalysts, as described herein. Subsequent to producing a solution of CO₂ hydration products, the solution may then be subjected to precipitation conditions to produce precipitation material comprising carbonates, bicarbonates, or a mixture thereof.

As shown in FIG. 1, a CO₂-containing gas from an industrial plant is sourced in step 130 and delivered to the processor to be processed optionally with the divalent cation-containing solution in processing step 120. The processing step (120) includes introducing CO₂ to a solution comprising catalyst, which, in some embodiments, may occur in a gas-liquid or gas-liquid-solid contactor. Such a solution, having been in contact with the CO₂-containing gas, produces CO₂ hydration products such as carbonic acid, bicarbonate, carbonate, or combinations thereof. As such, the solution in this step results in an increase in the CO₂ content of the solution (e.g., in the form of carbonic acid, bicarbonate, and/or carbonate), and a concomitant decrease in the amount of CO₂ in the CO₂-containing gas that is contacted with the water. When the CO₂-containing gas is put in contact with the catalyst optionally including the divalent cation-containing aqueous solution and optionally including the proton-removing agent, the solution may be alkaline such that the solution may have a pH 10 or lower, such as pH 9.5 or lower, including pH 9 or lower, for example, pH 8 or lower. Or between pH 7-12. The resultant solution may be acidic in some embodiments, having a pH of pH 6.0 or less, such as pH 5.0 or less, including pH 4.0 and less, for example, pH 3.0 or less. In some embodiments, the resultant solution is not acidic, the CO₂-charged solution having a pH 7 to pH 10, pH 7 to pH 9, pH 7.5 to pH 9.5, pH 8 to pH 10, pH 8 to pH 9.5, or pH 8 to pH 9. In some embodiments, the concentration of CO₂ in the CO₂-containing gas that is used to charge the aqueous solution is 10% or higher, 15% or higher, 25% or higher, including 50% or higher, such as 75% or higher, for example 85% or higher. In some embodiments, the amount of CO₂ in the CO₂-containing gas absorbed by the aqueous solution (e.g., solution optionally comprising divalent cations and/or proton-removing agents) is 99% or more; 95% or more; 90% or more; 85% or more; 80% or more; 75% or more; 70% or more; 65% or more; 60% or more; 55% or more; 50% or more; 45% or more; 40% or more; 35% or more; 30% or more; 25% or more; 20% or more; 15% or more; 10% or more; or 5% or more. In some embodiments, the amount of CO₂ in the CO₂-containing gas absorbed by the aqueous solution (e.g., solution optionally comprising divalent cations and/or proton-removing agents) is less than 99%; less than 95%; less than 90%; less than 85%; less than 80%; less than 75%; less than 70%; less than 65%; less than 60%; less than 55%; less than 50%; less than 45%; less than 40%; less than 35%; less than 30%; less than 25%; less than 20%; less than 15%; less than 10%; or less than 5%. In some embodiment, the amount of CO₂ in the CO₂-containing gas absorbed by the aqueous solution (e.g., solution optionally comprising divalent cations and/or proton-removing agents) is between 5% and 99%; between 10% and 95%; between 10% and 90%; between 10% and 85%; between 10% and 80%; between 10% and 75%; between 10% and 70%; between 10% and 65%; between 10% and 60%; between 10% and 55%; or between 10% and 50%.

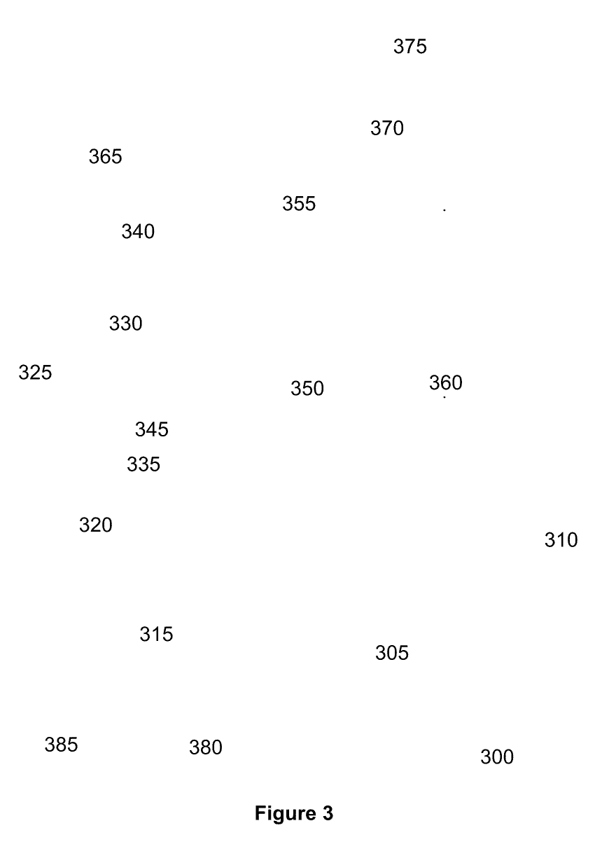

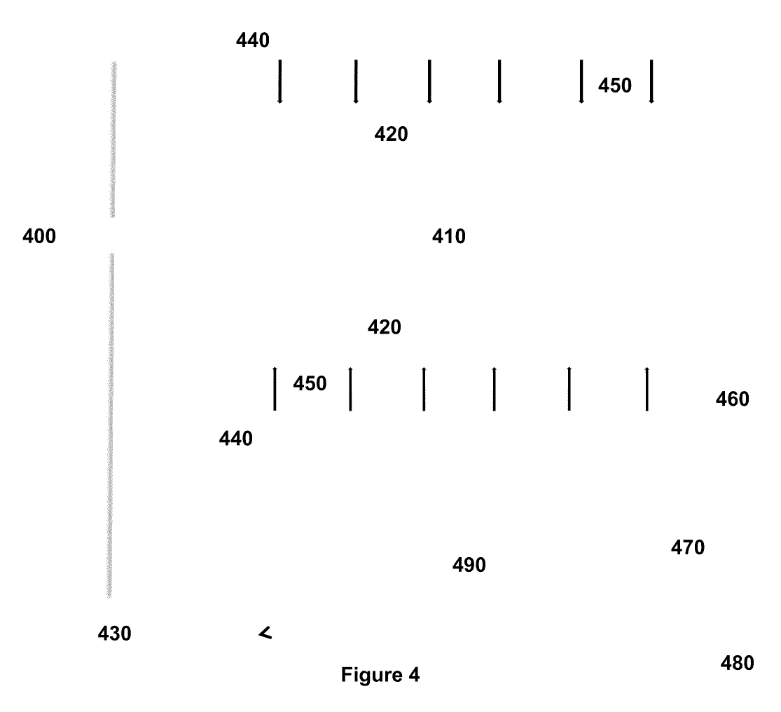

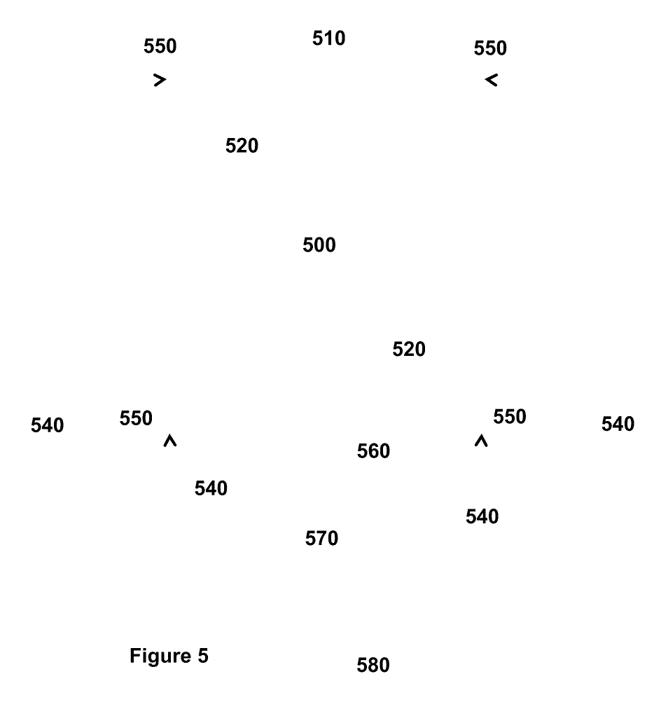

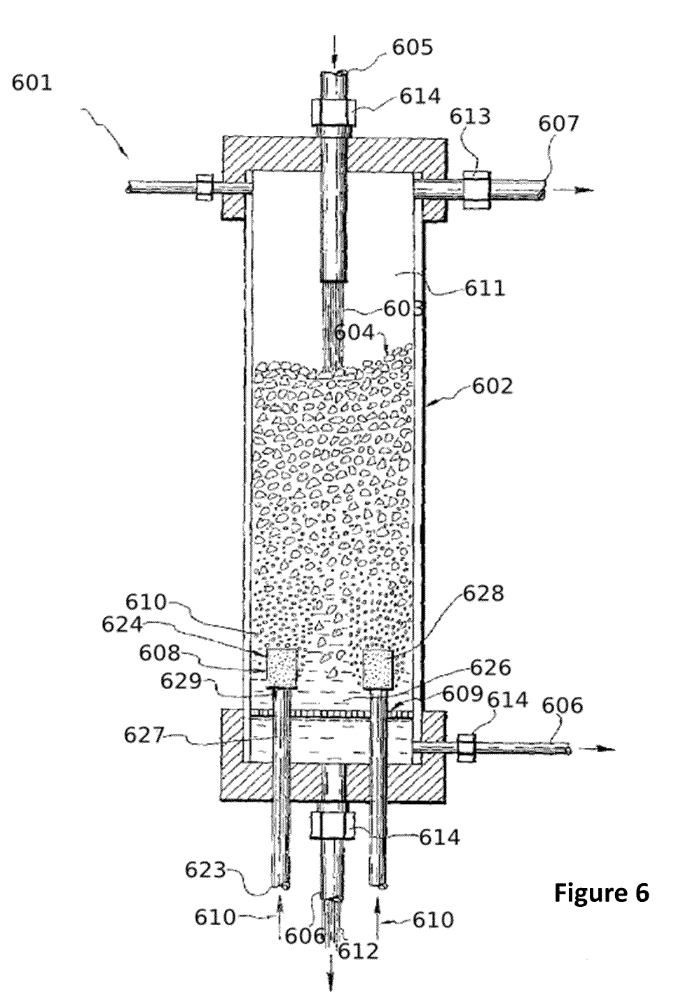

The CO₂-containing gas may be put in contact with the solution from one or more of the following positions: below, above, or at the surface level of the solid or the solution (e.g., catalyst optionally including alkaline earth metal cation-containing solution). Contact protocols include, but are not limited to, direct contacting protocols such as bubbling CO₂-containing gas through a volume of water, concurrent contacting means (i.e., contact between unidirectionally flowing gaseous and liquid phase streams), countercurrent means (i.e., contact between oppositely flowing gaseous and liquid phase streams), and the like. The CO₂-containing gas may be put in contact with the solid or the solution (e.g., catalyst optionally including divalent cation-containing solution) vertically (e.g., FIG. 3 and FIG. 6), horizontally (e.g., FIG. 4 and FIG. 5), or at some other angle. Contact may be accomplished through the use of infusers, bubblers, fluidic Venturi reactors, spargers, gas filters, sprayers, trays, catalytic bubble column reactors, draft-tube type reactors, packed column reactors, and the like, as may be convenient. Two or more (e.g., three or more, four or more, etc.) different gas-liquid of gas-liquid-solid contactors such as columns or other configurations may be employed, for example, in series or in parallel. Various means may be used to agitate or stir the solution to increase contact between CO₂ and the solution, wherein mechanical stirring, electromagnetic stirring, spinners, shakers, vibrators, blowers, ultrasonication, or the like may be used.

Contact of the solution of divalent cations comprising catalyst may be established with the CO₂-containing gas before, during, or before and during the time when the solution of divalent cations (or precipitation reaction mixture) is subjected to CO₂ precipitation conditions. Accordingly, embodiments of the invention include methods in which the solution of divalent cations comprising catalyst is contacted with a source of CO₂ prior to subjecting the resultant solution to precipitation conditions. Embodiments of the invention also include methods in which the precipitation reaction mixture is contacted with the source of CO₂ while the volume of precipitation reaction mixture is being subjected to precipitation conditions. Embodiments of the invention include methods in which the solution of divalent cations (or precipitation reaction mixture) comprising catalyst is contacted with the source of CO₂ both prior to subjecting the volume of water (e.g., alkaline earth metal ion-containing water) to carbonate and/or bicarbonate compound precipitation conditions and while the solution of divalent cations (or precipitation reaction mixture) is being subjected to precipitation conditions. Embodiments of the invention include methods in which the solution of divalent cations (or precipitation reaction mixture) is contacted with the aqueous solution including hydrated CO₂ (formed from the source of CO₂ and the catalyst optionally with the proton-removing agent) in carbonate and/or bicarbonate compound precipitation conditions. In some embodiments, the precipitation reaction mixture (e.g., supernatant of the precipitation reaction mixture) may be cycled more than once, wherein a first cycle of precipitation removes calcium and/or magnesium carbonate minerals, calcium and/or magnesium bicarbonate minerals, or a combination thereof, and leaves a solution to which metal ions, for example, alkaline earth metal ions such as Ca²⁺ and/or Mg²⁺ may be added. More CO₂ may be cycled through such a solution, precipitating more precipitation material comprising carbonate, bicarbonates, or mixtures thereof.

In addition to processing CO₂, embodiments of the invention also encompass processing other products resulting from combustion of carbon-based fuels. For example, at least a portion of one or more of NOx, SOx, VOC, mercury and mercury-containing compounds, or particulates that may be present in the CO₂-containing gas may be fixed (i.e., precipitated, trapped, etc.) in precipitation material. In some embodiments, the CO₂-containing gas may be processed before being used to charge the catalyst optionally including solution of divalent cations. For example, the CO₂-containing gas may be subjected to oxidation conditions to improve solubility of some of the components of the CO₂-containing gas, wherein oxidation conditions, for example, convert CO to CO₂, NO to NO₂, SO₂ to SO₃, and the like.

In addition to contacting the catalyst optionally with the divalent cation-containing solution with CO₂ in processing step 120, precipitation of precipitation material may occurs in step 120. CO₂ charging and precipitation of precipitation material may occur in the same unit or in different units of the processor. As such, in some embodiments, charging and precipitation may occur in the same unit. For example, precipitation may occur as the divalent cation-containing solution comprising catalyst is contacted with CO₂-containing gas (i.e., in gas-liquid contactor). In yet other embodiments of the invention, charging and precipitation may occur in separate units. For example, the divalent cation-containing solution comprising catalyst may first be charged with a CO₂-containing gas in a gas-liquid contactor, and then the resultant CO₂-charged solution may then be subjected to precipitation conditions in a precipitation reactor or vice versa.

Precipitation conditions used to invoke precipitation of precipitation material include those that modulate the physical and/or chemical environment of the precipitation reaction mixture to produce the desired precipitation material. For example, the temperature of the precipitation reaction mixture may be raised to a temperature suitable for precipitation of a desired carbonate and/or bicarbonate mineral. In such embodiments, the temperature of the water may be raised from 5° C. to 100° C., such as from 5 to 70° C., including 20° C. to 50° C., for example, from 25° C. to 45° C. As such, while a given set of precipitation conditions may have a temperature ranging from 0° C. to 100° C., the temperature may be raised in certain embodiments to produce the desired precipitation material. In some embodiments, the temperature is raised using energy generated from sources having low- or zero-carbon dioxide emissions (e.g., solar energy, wind energy, hydroelectric energy, etc.). In certain embodiments, excess and/or process heat (e.g., hot gas, steam, etc.) from the industrial plant carried in the CO₂-containing gas is employed to raise the temperature of the precipitation reaction mixture during precipitation. In some embodiments, contact of the divalent cation-containing solution with the CO₂-containing gas or the aqueous solution including hydrated CO₂ may raise the solution to the desired temperature, wherein, in some embodiments, the solution may need to be cooled to the desired temperature.

Certain additives may be added to the precipitation reaction mixture in order to influence the nature of the material (e.g., precipitation material) that is produced. For instance, certain additives may produce a more crystalline material over a more amorphous material. As such, in some embodiments, an additive is provided to the precipitation reaction mixture before or during the time when the precipitation reaction mixture is subjected to precipitation conditions. For example, certain polymorphs of calcium carbonate, which may precipitate in a number of different morphologies, are favored by trace amounts of certain additives. Without being limited by any theory, it is contemplated that vaterite, an unstable polymorph of CaCO₃, and which converts to calcite under appropriate conditions, may be obtained in high yields by including trace amounts of lanthanum salts (e.g., lanthanum chloride). Other transition metals and the like may be added to produce desired polymorphs. For instance, the addition of ferrous or ferric iron is known to favor the formation of disordered dolomite (protodolomite). Certain polymorphs may also be formed by providing seed crystals of the desired polymorph, or by providing some other template upon which the desired polymorph can form. In some embodiments, additives, seed crystals, and the like are used to produce material that is relatively crystalline (e.g., >90% crystalline) or substantially crystalline (e.g., >95% crystalline) in order to reduce energy requirements associated with drying amorphous material, which amorphous material generally comprises water in addition to waters of hydration. As such, in some embodiments, methods of the invention comprise contacting a solution including divalent cations with a CO₂-containing gas or the aqueous solution including hydrated CO₂; producing from the solution (e.g., by seeding) a crystalline material comprising carbonate, bicarbonates, or mixtures thereof; and separating the crystalline material from the solution. In some embodiments, and under certain conditions, energy requirements associated with drying material are reduced or eliminated by utilizing a method for producing material of the invention that uses little if any water.

In normal seawater, 93% of the dissolved CO₂ is in the form of bicarbonate (HCO₃⁻) and 6% is in the form of carbonate (CO₃²⁻). When calcium carbonate precipitates from normal seawater, CO₂ is released. In freshwater above pH 10.33, greater than 90% of the dissolved CO₂ is in the form of carbonate, and no CO₂ is released during precipitation of calcium carbonate. In seawater this transition occurs at a slightly lower pH, closer to a pH of pH 9.7. While pH of the precipitation reaction mixture may range from pH 5 to pH 14 during a given precipitation process, the pH may be raised to alkaline levels in some embodiments in order to drive precipitation of precipitation material comprising carbonates, as well as other compounds (e.g., bicarbonates, hydroxide compounds, etc.). In some embodiments, pH is raised to a level that minimizes if not eliminates CO₂ production during precipitation, causing dissolved CO₂ (e.g., in the form of carbonate, bicarbonate, or a mixture thereof) to be trapped in precipitation material. In these embodiments, pH may be raised to pH 9 or higher, such as pH 10 or higher, including pH 11 or higher, for example, pH 12 or higher. In such embodiments, the pH may be raised using proton-removing agents or methods for effecting proton removal.

As summarized above, pH of the divalent cation-containing solution may be raised using any convenient approach. In some embodiments, a pH-modifying agent (e.g., a proton-removing agent) may be employed, examples of which include agents such as oxides (e.g., calcium oxide, magnesium oxide), hydroxides (e.g., potassium hydroxide, sodium hydroxide, brucite (Mg(OH)₂), Ca(OH)₂, etc.), carbonates (e.g., sodium carbonate), and the like.

The processing step may further comprise additional units. For example, mineral processing may be achieved in a separate mineral processing unit. As described in further detail below, the processor (e.g., processor 220 in FIG. 2) may include any of a number of different components (e.g., temperature control components) for controlling precipitation conditions and the like. Such components may be used to, for example, heat the precipitation reaction mixture to a desired temperature; introduce chemical additives (e.g., proton-removing agents such as KOH, NaOH); operate electrochemical components (e.g., cathodes/anodes), gas-charging components, and/or pressurization components (e.g., for operating under pressurized conditions, such as from 50 psi to 800 psi, 100 psi to 800 psi, 400 psi to 800 psi, or any other suitable pressure range); agitate or stir the precipitation reaction mixture (e.g., mechanical agitation or physical stirring to re-circulate industrial plant flue gas through the precipitation plant). The processing step may further include any of a number of different steps that allow for monitoring (e.g., inline monitoring) one or more parameters such as internal reactor pressure, pH, precipitation material particle size, metal-ion concentration, conductivity of the aqueous solution, alkalinity of the aqueous solution, and pCO₂. Monitoring conditions during the carbonate and/or bicarbonate precipitation process may allow for corrective adjustments to be made during the precipitation process. For example, corrective adjustments may be made to increase or decrease precipitation rates of precipitation material.

As illustrated in FIG. 1, slurry comprising precipitation material resulting from processing step 120 may be concentrated in step 140 to produce a slurry concentrated in precipitation material. In some embodiments, the slurry including the precipitation material resulting from processing step 120 is used as is, as a cementitious composition. In some embodiments, the slurry may be further processed to result in the composition of the invention. In some embodiments, precipitation material is separated from precipitation reaction mixture in step 140 to produce a dewatered precipitation material. Separation of the precipitation material from the precipitation reaction mixture is achieved using any of a number of convenient approaches, including draining (e.g., gravitational sedimentation of the precipitation material followed by draining), decanting, filtering (e.g., gravity filtration, vacuum filtration, filtration using forced air), centrifuging, pressing, or any combination thereof. In some embodiments, precipitation material is separated from precipitation reaction mixture by flowing precipitation reaction mixture against a baffle, against which supernatant deflects and separates from particles of precipitation material, which precipitation material is collected in a collector. In some embodiments, precipitation material is separated from precipitation reaction mixture by flowing precipitation reaction mixture in a spiral channel separating particles of precipitation material and collecting the precipitation material from an array of spiral channel outlets. Mechanically, at least one liquid-solid separation apparatus is operably connected to the processor (e.g., processor 220 of FIG. 2) such that precipitation reaction mixture may flow from the processor to the liquid-solid separation apparatus. The precipitation reaction mixture may flow directly to the liquid-solid separation apparatus, or the precipitation reaction mixture may be pre-treated (e.g., coarse filtration) to remove large-sized particles of precipitation material from the precipitation reaction mixture prior to providing the precipitation reaction mixture to the liquid-solid separation apparatus as large-sized particles may interfere with the liquid-solid separation apparatus or process.

Energy requirements for any of the foregoing separation approaches may be fulfilled by adapting the approach to utilize any of a number of energy-containing waste streams (e.g., waste heat from waste gas streams) provided by industrial plants; however, it will be appreciated by a person having ordinary skill in the art that separation approaches requiring less energy are desirable in terms of lessening the carbon footprint of the invention.

Concentration of the precipitation material in the precipitation reaction mixture or separation of the precipitation material from the precipitation reaction mixture may be achieved with a single liquid-solid separation apparatus. In some embodiments, a combination of two, three, four, five, or more than five liquid-solid separation apparatus may be used. Combinations of liquid-solid separators may be used in series, parallel, or in combination of series and parallel depending on desired throughput. Furthermore, as with methods and systems of the invention in general, concentration and/or separation may be achieved continuously, semi-batch wise, or batch wise with methods and liquid-solid separation apparatus of the invention. In some embodiments, liquid-solid separation apparatus or combinations thereof are used to process precipitation reaction mixture at 100 L/min to 2,000,000 L/min, 100 L/min to 1,000,000 L/min, 100 L/min to 500,000 L/min, 100 L/min to 250,000 L/min, 100 L/min to 100,000 L/min, 100 L/min to 50,000 L/min, 100 L/min to 25,000 L/min, and 100 L/min to 20,000 L/min. In some embodiments, liquid-solid separation apparatus or combinations thereof are used to process precipitation reaction mixture at 1000 L/min to 2,000,000 L/min, 5000 L/min to 2,000,000 L/min, 10,0000 L/min to 2,000,000 L/min, 20,000 L/min to 2,000,000 L/min, 25,000 L/min to 2,000,000 L/min, 50,000 L/min to 2,000,000 L/min, 100,000 L/min to 2,000,000 L/min, 250,000 L/min to 2,000,000 L/min, 500,000 L/min to 2,000,000 L/min, and 1,000,000 L/min to 2,000,000 L/min. In some embodiments, liquid-solid separation apparatus or combinations thereof are used to process precipitation reaction mixture at 1000 L/min to 20,000 L/min, 5000 L/min to 20,000 L/min, 10,000 L/min to 20,000 L/min, 1000 L/min to 10,000 L/min, 2000 L/min to 10,000 L/min, 3000 L/min to 10,000 L/min, 4000 L/min to 10,000 L/min, 5000 L/min to 10,000 L/min, 6000 L/min to 10,000 L/min, 7000 L/min to 10,000 L/min, 8000 L/min to 10,000 L/min, 9000 L/min to 10,000 L/min, or 9500 L/min to 10,000 L/min.

Combinations of liquid-solid separators in series, parallel, or series and parallel may also be used to increase separation efficiencies. In addition, supernatant resulting from a liquid-solid separation apparatus or an assembly of liquid-solid separation apparatus may be recirculated through the liquid-solid separation apparatus or assembly of liquid-solid separation apparatus to increase separation efficiency. In some embodiments, 30% to 100%, 40% to 100%, 50% to 100%, 60% to 100%, 70% to 100%, 75% to 100%, 80% to 100%, 85% to 100%, 90% to 100%, 95% to 100%, 96% to 100%, 97% to 100%, 98% to 100%, 99% to 100% of precipitation material is collected from the precipitation reaction mixture. Depending upon the amount of precipitation material removed from the precipitation reaction mixture, the supernatant may be delivered back to the processor or provided to an electrochemical apparatus of the invention. Also, a portion of the supernatant may be delivered back to the processor and a portion of the supernatant may be provided to the electrochemical apparatus, wherein distribution of the supernatant is determined based upon manufacturing needs. The supernatant, or portion thereof, provided to the processor, electrochemical apparatus, or processor and electrochemical apparatus is optionally pre-treated. For example, supernatant for use in the electrochemical apparatus may need to be filtered to remove particulate matter and divalent cations. In some embodiments, supernatant with a relatively high concentration of precipitation material is delivered back to the processor (e.g., precipitation reactor) for agglomeration of precipitation material particles. In some embodiments, supernatant with a relatively high concentration of dissolved divalent cations (e.g., Ca²⁺ or Mg²⁺) is delivered back to the processor as a source of divalent cations. In some embodiments, supernatant with a relatively low concentration of precipitation material and dissolved divalent cations may be provided to an electrochemical system of the invention.

In some embodiments, the precipitation material is not separated, or is only partially separated, from the precipitation reaction mixture. In such embodiments, the precipitation reaction mixture, including some (e.g., after passing through a liquid-solid separation apparatus) or all of the precipitation material, may be disposed of in any of a number of different ways. In some embodiments, the precipitation reaction mixture, including some or all of the precipitation material, is transported to a land- or water-based location and deposited at the location. Transportation to the ocean is especially useful when the source of divalent cations is seawater. It will be appreciated that the carbon footprint, amount of energy used, and/or amount of CO₂ produced for sequestering a given amount of CO₂ from an industrial exhaust gas is minimized in a process where no further processing of the precipitation material occurs beyond disposal. In some embodiments, precipitation material, or a slurry comprising the precipitation material, may simply be transported to a location for long-term storage, effectively sequestering CO₂. For example, the precipitation material may be transported and placed at long-term storage sites, wherein such sites are above ground, below ground, deep in the ocean, and the like. In these embodiments, wherein the precipitation material is transported to a long-term storage site, it may be transported in empty conveyance vehicles (e.g., barges, train cars, trucks, etc.) that were employed to transport the fuel or other materials to the industrial plant and/or precipitation plant. In this manner, conveyance vehicles used to bring fuel to the industrial plant, materials to the precipitation plant (e.g., alkali sources) may be employed to transport precipitation material, and therefore sequester CO₂ from the industrial plant. As desired, compositions made up of a slurry comprising the precipitation material may be stored for a period of time following precipitation and prior to disposal. For example, the composition may be stored for a period of time ranging from 1 to 1000 days or longer, such as 1 to 10 days or longer, at a temperature ranging from 1° C. to 40° C., such as 20° C. to 25° C.

In the embodiment illustrated in FIG. 1, concentrated or separated precipitation material (e.g., “wet cake”) is dried in a drying step (160) to produce a dried precipitation material. Drying may be achieved by air-drying the wet cake of precipitation material. Where the wet cake is air dried, air-drying may be at room temperature or at an elevated temperature. In some embodiments, the CO₂-containing gas from the industrial plant provides elevated temperatures. In such embodiments, the CO₂-containing gas (e.g., flue gas) from the power plant may first be used in drying step 160, wherein the CO₂-containing gas may have a temperature ranging from 30° C. to 700° C., such as 75° C. to 300° C. The CO₂-containing gas may be contacted directly with the wet cake of precipitation material, or it may be used to indirectly heat gases (e.g., air). The desired temperature may be provided by the CO₂-containing gas by having a gas conveyer (e.g., duct) from the industrial plant originate from a suitable location, for example, from a location a certain distance in a heat recovery steam generator (HRSG) or up a flue, as determined based on the specifics of the exhaust gas and configuration of the industrial plant. In yet another embodiment, precipitation material is spray dried to dry the precipitation material, wherein a slurry comprising the precipitation material is dried by feeding it through a hot gas (e.g., CO₂-containing gas from the industrial plant, or a gas such as air heated by the CO₂-containing gas), and further wherein the slurry is pumped through an atomizer into a main drying chamber and hot gas is passed as a co-current or counter-current to the atomizer direction. In certain embodiments, drying is achieved by freeze-drying (i.e., lyophilization), wherein the precipitation material is frozen, the surrounding pressure is reduced, and enough heat is added to allow for sublimation of frozen water. Depending upon the particular drying protocol, drying may further include filtration through a filtration element, freeze-drying by means of a freeze-drying structure, spray drying by means of a spray-drying structure, and the like.

Wet cake comprising precipitation material may be washed in a washing step (150) before drying, as illustrated at optional step 150 of FIG. 1. The wet cake may be washed with freshwater to remove, for example, salts such as NaCl from the wet cake of precipitation material. Used wash water may be processed by any convenient means, for example, disposed of in a tailings pond, or reused in some portion of the process.

In some embodiments, precipitation material is refined in some manner prior to subsequent use. Refinement as illustrated in step 170 of FIG. 1 may include a variety of different protocols. In some embodiments, precipitation material is subjected to mechanical refinement (e.g., grinding) in order to obtain a product with desired physical properties (e.g., particle size, etc.). The composition obtained after the processing of the precipitation material may be used as a cement composition. The cement composition may be a self-cement or a hydraulic cement or may be used as a supplementary cementitious material. In some embodiments, one or more components may be added to the precipitation material. In such embodiments, where the precipitation material is to be employed as a cement, one or more additives such as sands, aggregates, supplementary cementitious materials, etc., may be added to produce the final product (e.g., concrete or mortar). For example, the precipitation material may be combined with a hydraulic cement, wherein the precipitation material is used as a supplementary cementitious material, for example, as a sand, a gravel, an aggregate, etc.).

Precipitation material or the composition produced by methods of the invention may be used in an article of manufacture. In other words, the precipitation material or the composition may be used in some embodiments to make a manufactured item. The precipitation material or the composition may be employed alone or in combination with one or more additional materials such that the precipitation material or the composition is a component of the manufactured item. Manufactured items may vary, wherein examples of manufactured items that may be produced with methods of the invention include building materials and non-building materials such as non-cementitious manufactured items. Building materials include components of concrete such as cement, aggregate (both fine and coarse), supplementary cementitious materials, and the like. Building materials of interest also include pre-formed building materials, which vary greatly, and may include molded, cast, cut, or otherwise produced into a structure with a defined physical shape. Pre-formed building materials are distinct from amorphous building materials (e.g., particulate compositions such as powder) that do not have a defined and stable shape, but instead conform to the container in which they are held (e.g., a bag or other container). Illustrative pre-formed building materials include, but are not limited to, bricks, boards, conduits, beams, basins, columns, drywalls, and the like. Further examples and details regarding formed building materials include those described in U.S. Provisional Patent Application Nos. 61/110,489, filed on 31 Oct. 2008, and 61/149,610, filed 3 Feb. 2009, each of which is titled “CO₂-sequestering Formed Building Materials,” and each of which is incorporated herein by reference. In certain embodiments, the precipitation material is utilized to produce aggregates. Such aggregates, methods for their manufacture and use are described in co-pending U.S. patent application Ser. No. 12/475,378, filed 29 May 2009, titled “Rock and Aggregate, and Methods of Making and Using the Same,” the disclosure of which is herein incorporated by reference. Examples of using the product in a building material include instances where the product is employed as a construction material for some type of manmade structure, e.g., buildings (both commercial and residential), roads, bridges, levees, dams, and other manmade structures etc. The building material may be employed as a structure or nonstructural component of such structures.

In some embodiments, the aqueous solution including the hydrated CO₂ (e.g., carbonates and/or bicarbonates); the slurry obtained after the reaction of the hydrated CO₂ with the aqueous solution including divalent cations; and/or the precipitation material or supernatant obtained after separation of the precipitation material from the slurry, may be injected underground for storage or disposal. In some embodiments, there is provided a method including contacting a gaseous stream comprising CO₂ with a catalyst to form a solution comprising hydrated CO₂; treating the solution with a proton-removing agent; and injecting the solution underground.

As such, the invention provides methods for geological sequestration of carbon dioxide in a subterranean site. These subterranean sites include, but are not limited to, saline aquifers, petroleum reservoirs, deep coal seams, sub-oceanic formations, and the like. In some embodiments, the subterranean site may contain water with greater than 1,000 ppm; 2,500 ppm; 5,000 ppm; 7,500 ppm; 10,000 ppm; 25,000 ppm; 50,000 ppm; or 100,000 ppm total dissolved solids. In some embodiments, the subterranean site may contain water with less than 100,000 ppm; 50,000 ppm; 25,000 ppm; 10,000 ppm; 7,500 ppm; 5,000 ppm; 2,500 ppm; or 1,000 ppm total dissolved solids. In some embodiments, the subterranean site may contain water between 1,000 and 100,000 ppm; 1,500 ppm and 50,000 ppm; 1,500 ppm and 25,000 ppm; or 1,500 ppm and 10,000 ppm total dissolved solids. The capacity of a subterranean site containing an aqueous solution (e.g., an aquifer or petroleum reservoir) may be increased by removal of the aqueous solution from the subterranean site. The aqueous solution may then become a source of divalent cations or proton-removing agents for processing hydrated CO₂ species (e.g., carbonates and/or bicarbonates) as described herein. Hydrated CO₂ species processed with aqueous solution from a subterranean site may subsequently be injected into the subterranean site whence the aqueous solution came; returned to a different subterranean site; formed into solids for use as building materials or other products as described herein, optionally injecting separated supernatant to the same or a different subterranean site; or some combination thereof. In some embodiments, injectate (i.e., an aqueous solution comprising hydrated CO₂ such as carbonates and/or bicarbonates), optionally treated with divalent cations and/or proton-removing agents, is injected into a subterranean site under conditions in which nahcolite does not precipitate (i.e., below nahcolite solubility).

Injection of solutions comprising carbonates and/or bicarbonates addresses many of the issues associated with conventional carbon capture and sequestration (“CCS”), which concerns capture of CO₂ and storage as supercritical carbon dioxide in geological formations. First, the costs of separating CO₂ from a waste stream comprising CO₂, compressing the CO₂, and transporting the compressed CO₂ are greatly reduced when the methods provided herein are compared with conventional CCS. Second, risks associated with underground storage are also alleviated. Over very long time periods (typically tens, hundreds, or even thousands of years), it is thought that CO₂ injected in conventional CCS processes will “mineralize” into bicarbonates and/or carbonates. These more stable forms of carbon reduce the risks associated with leaks from underground formations. In methods provided herein, at least a portion (if not all) of any injected CO₂ may already be in one of the more stable ionic forms (e.g., carbonates and/or bicarbonates), reducing overall risk when compared to conventional CCS. These more stable forms also may make viable certain subterranean sites, which would otherwise be unsuitable for sequestration of supercritical carbon dioxide. For example, in some embodiments, the subterranean site for injection may be at least 100 m; 250 m; 500 m; 1000 m; 2500 m; 5000 m; 10,000 m; 15,000 m; or 25,000 m below ground level. For example, in some embodiments, the subterranean site for injection may be less than 25,000 m; 15,000 m; 10,000 m; 5,000 m; 2,500 m; 1,000 m; 500 m; 250 m; or 100 m below ground level. For example, in some embodiments, the subterranean site for injection may be between 100 m and 15,000 m; 100 m and 10,000 m; 100 m and 5,000 m; 250 m and 15,000 m; 250 m and 10,000 m; 250 m and 5,000 m; 500 m and 15,000 m; 500 m and 10,000 m; or 500 m and 5,000 m. In some embodiments, cap rock is not necessary above a subterranean site in which a carbonate and/or bicarbonate composition has been injected.

Porosity as used herein includes the fraction of void space in the material, where the void may contain, for example, air or water. It may be defined by the ratio Vᵥ/Vₜ=φ, where Vᵥ is the volume of void-space (such as fluids) and Vₜ is the total or bulk volume of material, including the solid and void components. Porosity may be between 0% and 100%, typically ranging from less than 1% for solid granite to more than 50% for peat and clay. In some embodiments, a storage site for injection of hydrated CO₂, optionally further treated with proton-removing agents and/or divalent cations, may have a porosity of greater than 1%, 5%, 10%, 20%, 30%, 40%, 50%, 60%, 70%, 80%, 90%, or 95%. In some embodiments, a storage site for injection of hydrated CO₂, optionally further treated with proton-removing agents and/or divalent cations, may have a porosity of less than 100%, 95%, 90%, 80%, 70%, 60%, 50%, 40%, 30%, 20%, 10%, or 5%. In some embodiments, a storage site for injection of hydrated CO₂, optionally further treated with proton-removing agents and/or divalent cations, may have a porosity of between 1% and 100%, 5% and 95%, 10% and 90%, 20% and 80%, 20% and 70%, 20% and 60%, 20% and 50. In some embodiments, a subterranean site for injection of hydrated CO₂, optionally further treated with proton-removing agents and/or divalent cations may be substantially free of cap rock or comprise a cap rock unsuitable for CCS. In some embodiments, a subterranean site for injection of hydrated CO₂, optionally further treated with proton-removing agents and/or divalent cations may be a subterranean site that is unsuitable for sequestration of supercritical CO₂. The subterranean site may be unsuitable for storage of supercritical CO₂ due to the presence of porous or fractured cap rock. “Cap rock” as used herein includes gas or supercritical fluid-impermeable rock that confines reservoirs and prevents the migration or leakage of reservoir hydrocarbons, gases, or supercritical fluids.

In some embodiments, methods provided herein include increasing the capacity of a subterranean site by removal of an aqueous solution from the subterranean site and, optionally, absorption of CO₂ into at least a portion of the aqueous solution and conversion of the CO₂ into bicarbonates and/or carbonates. In some embodiments, aqueous solution removed from a subterranean site comprises divalent cations such as calcium, magnesium, strontium, and the like. In some embodiments, aqueous solution removed from a subterranean site comprises one or more proton-removing species and possess an amount of alkalinity as measured in mEq/L (milliequivalent per liter). At least a portion of the proton removing species may be used to form hydrated CO₂ (e.g., bicarbonates and/or carbonates) as described herein. The removal of the aqueous solution from the subterranean site may increase the capacity of the subterranean site for additional carbon storage either as supercritical CO₂ from conventional CCS or as an aqueous solution comprising bicarbonates and/or carbonates. In some embodiments, the bicarbonates and/or carbonates are returned to the same subterranean site whence the aqueous solution from the subterranean site was removed. In some embodiments, the bicarbonates and/or carbonates are returned to a different subterranean site whence the aqueous solution from the subterranean site was removed. In some embodiments, an aqueous solution comprising carbonates and/or bicarbonates is injected into a subterranean site using the same well bore that was used to draw aqueous solution from the subterranean site. In some embodiments, a different well bore is used. In some embodiments, a portion of hydrated CO₂ (e.g., bicarbonates and/or carbonates) may be converted to mineralized (solid) forms outside of the subterranean location (e.g., in a system described herein).

### Systems

Systems of the invention may have any configuration that enables practice of the particular production method of interest, which methods are primarily described above.

FIG. 2 provides a schematic of a system (200) of the invention. System 200 of FIG. 2 includes CO₂-containing gas source 230 (e.g., coal-fired power plant). This system also includes a conveyance structure such as a pipe, duct, or conduit, which directs the CO₂-containing gas to processor 220 from CO₂-containing gas source 230. Also shown in FIG. 2 is divalent cation-containing solution source 210 (e.g., body of water, tank of divalent cation-containing solution, etc.). In some embodiments, divalent cation-containing solution source 210 includes a conveyance structure such as a pipe, duct, or conduit, which directs the divalent cation-containing solution (e.g., alkaline earth metal ion-containing aqueous solution) to the processor (220) or in some embodiments, to the aqueous solution including hydrated CO₂. Where the divalent cation-containing solution source is seawater, the conveyance structure is in fluid communication with the source of seawater (e.g., the input is a pipe line or feed from ocean water to a land-based system, or the input is an inlet port in the hull of ship in an ocean-based system). Although not shown, system 200 further comprise a source of proton-removing agents and a source of catalysts. In some embodiments, as described herein, the catalyst is integrated with the processor (220). Various embodiments of the catalyst are described herein. In some embodiments, the proton-removing agent may also be integrated with the processor 220.

In some embodiments, the processor is operably connected to a reactor (not shown in FIG. 2) and the source of divalent cations is connected to the reactor such that the solution including the hydrated CO₂ (formed after the contact of the gaseous stream of CO₂ with the catalyst in the processor optionally in the presence of the proton-removing agent) is transferred from the processor to the reactor for the treatment of the hydrated CO₂ with the divalent cations to form the precipitated material. The supernatant formed from the precipitated material may be re-circulated back to the reactor after the separation of the precipitated material.

The aqueous solution of divalent cations provided to the processor or a component thereof (e.g. gas-liquid contactor, gas-liquid-solid contactor; etc.) may be recirculated by a recirculation pump such that absorption of CO₂-containing gas (e.g., comprising CO₂, SOx, NOx, metals and metal-containing compounds, etc.) is optimized within a gas-liquid contactor or gas-liquid-solid contactor within the processor. With or without recirculation, processors of the invention or a component thereof (e.g. gas-liquid contactor, gas-liquid-solid contactor; etc.) may be configured to effect at least 25%, or at least 30%, or at least 45%, or at least 50%, or at least 60%, or at least 70%, or at least 80%, or at least 90%, or between 20-100%, or between 30-100%; or between 40-100%, or between 50-100%, or between 60-100%, or between 70-100%; or between 80-100%; or between 90-100%, or between 20-95%, or between 30-95%; or between 40-95%, or between 50-95%, or between 60-95%, or between 70-95%; or between 80-95%; or between 90-95%, or between 20-85%, or between 30-85%; or between 40-85%, or between 50-85%, or between 60-85%, or between 70-85%; or between 20-75%, or between 30-75%; or between 40-75%, or between 50-75%, or between 60-75%, or between 20-65%, or between 30-65%; or between 40-65%, or between 50-65%, or between 60-65%, or between 20-55%, or between 30-55%; or between 40-55%, or between 50-55%, dissolution of the CO₂ from the CO₂-containing gas. Dissolution of other gases (e.g., SOx) may be even greater, for example, at least 95%, 98%, or 99%.

Additional parameters that provide optimal absorption of CO₂-containing gas include a specific surface area of 0.1 to 30, 1 to 20, 3 to 20, or 5 to 20 cm⁻¹; a liquid side mass transfer coefficient (k_{L}) of 0.05 to 2, 0.1 to 1, 0.1 to 0.5, or 0.1 to 0.3 cm/s; and a volumetric mass transfer coefficient (K_{L}a) of 0.01 to 10, 0.1 to 8, 0.3 to 6, or 0.6 to 4.0 s⁻¹.

In some embodiments, when the catalytic absorption of CO₂-containing gas is by the catalyst optionally present in a solution including the proton-removing agent, it causes formation of an aqueous solution including hydrated CO₂. In some embodiments, the aqueous solution including hydrated CO₂ may then be treated with an aqueous solution containing the divalent cation to form the precipitation material. In some embodiments, the precipitation material is formed inside the processor. In some embodiments, the aqueous solution including hydrated CO₂ is transferred out of the processor to another container where the aqueous solution containing the divalent cation is added to form the precipitation material. The processor, while providing a structure for precipitation of precipitation material, may also provide a preliminary means for settling (i.e., the processor may act as a settling tank). The processor, whether providing for settling or not, may provide a slurry of precipitation material to a dewatering feed pump, which, in turn, provides the slurry of precipitation material to the liquid-solid separator where the precipitation material and the precipitation reaction mixture are separated.

In some embodiments, when the catalytic absorption of CO₂-containing gas is by the aqueous solution of divalent cations, it causes precipitation of at least a portion of precipitation material in the gas-liquid contactor. In some embodiments, precipitation primarily occurs in a precipitator of the processor. The processor, while providing a structure for precipitation of precipitation material, may also provide a preliminary means for settling (i.e., the processor may act as a settling tank). The processor, whether providing for settling or not, may provide a slurry of precipitation material to a dewatering feed pump, which, in turn, provides the slurry of precipitation material to the liquid-solid separator where the precipitation material and the precipitation reaction mixture are separated.

In some embodiments, the invention provides processors comprising a gas-liquid or gas-liquid-solid contactor, which may contain one or more catalysts capable of catalyzing the hydration of dissolved CO₂ into aqueous bicarbonate and/or carbonate. The catalyst (e.g., enzyme) may be free or immobilized on a support suitable for the catalyst. In some embodiments, for example, the gas-liquid contactor or gas-liquid contactor may comprise a packed column, packed tower, spray tower, or aspersion tower configured to accept catalyst as described herein. Such embodiments allow for smaller sized gas-liquid contactors due to increased CO₂ absorption efficiency. The gas-liquid or gas-liquid-solid contactor may be operably connected to a precipitator of the processor, or in embodiments in which the gas-liquid or gas-liquid-solid contactor is configured to produce a precipitation material upon contact with the CO₂-containing gas, the gas-liquid or gas-liquid-solid contactor may be operably connected to a liquid-solid separation apparatus, which apparatus are described in further detail below. In any case, the processor comprises a liquid outlet for discharging the composition (e.g., solution, slurry, etc.) resulting from processing the CO₂-containing gas in the processor.

In some embodiments, the gas-liquid or gas-liquid-solid contactor is configured to receive CO₂-containing gas from the CO₂-containing gas source, optionally pre-cooled by means of an operably connected heat exchanger. A spray tower of the invention may comprise a multitude of stages and/or spray inlets (e.g., nozzles) at various locations throughout the tower. As such, the spray tower may be a multi-stage spray tower such as a dual-stage spray tower. Such spray towers are described in U.S. Provisional Patent Application 61/223,657, filed 7 Jul. 2009, titled “Gas, Liquid, Solid, Contacting: Methods and Apparatus,” the disclosure of which is incorporated herein by reference. The spray tower may also be configured as a packed tower or another type of tower known in the art. Operationally, spray towers of the invention are configured to spray a solution (e.g., aqueous solution of divalent cations such as seawater and/or brine and/or recirculated water and/or fresh water and/or water containing the proton-removing agent) into a medium comprising a CO₂-containing containing gas (e.g., atmosphere comprising CO₂-containing gas; solution comprising CO₂-containing gas; immobilization material comprising catalyst and CO₂-containing gas; combinations thereof, and the like).

The spray tower may be equipped with one or more catalysts for catalyzing the hydration of CO₂ (e.g., to produce bicarbonate and/or carbonate). In some embodiments, at least one catalyst is a biocatalyst such as an enzyme (e.g., carbonic anhydrase). The enzyme may be immobilized on an immobilization material such as an immobilization material described herein, which immobilization material may be configured to promote the hydration of CO₂. For example, such an immobilization material may allow CO₂-containing gas or a solution comprising dissolved CO₂ to freely pass through the immobilization material such that the conversion of CO₂ by an immobilized catalyst is minimally affected by the rate of diffusion. Such an immobilization material may also allow a solution comprising bicarbonate and/or carbonate to freely pass through the immobilization material, which property is also desirable as bicarbonate and/or carbonate may inhibit an enzyme such as carbonic anhydrase. Immobilization material may also be configured to allow certain additives (e.g., Zn²⁺) in solution to freely pass through the immobilization material, which additives may activate an enzyme such as carbonic anhydrase. The spray tower may also be configured to contain one or more catalysts in addition to an enzyme (e.g., carbonic anhydrase), or to the exclusion of an enzyme. Such catalysts are described above, and such catalysts may be immobilized on immobilization material or an interior surface of the spray tower.

A multi-stage spray tower of the invention may be configured to allow for CO₂-containing gas to enter the bottom of the spray tower where it may be absorbed and further cooled (if needed) by a solution of divalent cations (e.g., seawater, brine, etc.) or an aqueous solution optionally including a proton-removing agent, sprayed into the spray tower through spray inlets (e.g., nozzles). Owing to this general configuration, the bottom or lowest stage of a multi-stage spray tower is generally hotter than the top or highest stage of the multi-stage spray tower. As such, in some embodiments, the bottom stage is not configured to comprise a biocatalyst (e.g., an enzyme such as carbonic anhydrase), especially if the temperature would destroy the biocatalyst. If the CO₂-containing gas is pre-cooled to an adequately low temperature, then the bottom stage may be configured to comprise a biocatalyst. In some embodiments, the bottom stage may be configured to comprise a catalyst other than a heat-sensitive biocatalyst. For example, the bottom stage may be configured to comprise an organic or inorganic catalyst. Depending on conditions (e.g., temperature), each stage of a multi-stage spray tower may be configured to comprise a different catalyst. In some embodiments, the multi-stage spray tower comprises stages configured to not contain a catalyst. In some embodiments, as the CO₂-containing gas travels through multiple stages of the multi-stage spray tower, the spray tower sprays a divalent cation-containing solution or an aqueous solution optionally including a proton-removing agent when CO₂ therein becomes more soluble in the divalent cation-containing solution or an aqueous solution optionally including a proton-removing agent, respectively. The resulting CO₂-charged solution collects in collection areas of the multi-stage spray tower, which collection areas are in fluid communication with a precipitator or liquid-solid separator depending upon inputs (e.g., concentration of divalent cation-containing solution; concentration of proton-removing agents) and conditions (e.g., temperature) under which CO₂-containing gas is processed.

FIG. 3 shows an embodiment of a gas-liquid-solid contactor of the invention. In such gas-liquid-solid contactors, a contacting chamber is configured to allow for an immobilization material comprising catalyst, a chamber for physically separating catalyst, or a combination thereof. Such gas-liquid-solid contactors are configured to allow for a proton-removing agent slurry comprising a liquid (e.g., a source of divalent cations such as seawater, brine, etc. or an aqueous solution optionally including a proton-removing agent) and a solid component (e.g., a proton-removing agent such as Mg(OH)₂, fly ash, cement kiln dust, etc.) to enter the contacting chamber through an inlet conduit (300) and mix with product slurry (i.e., proton-removing agent slurry that has contacted CO₂-containing gas) in a reservoir (305). In some embodiments, a screw conveyor (310) provides comminution and mixing of the proton-removing agent slurry with the CO₂-containing gas as it enters the contacting chamber. In some embodiments, the CO₂-containing gas enters the contacting chamber through an inlet without first mixing with the proton-removing agent slurry. In some embodiments, the gas-liquid-solid contactor comprises at least two levels, or sections, of bidirectional droplet- or stream-producing arrays (350 and 355) (e.g., sprays) with conduits (360) for the CO₂-containing gas to travel upwards through the gas-liquid contactor. As shown in FIG. 3, a slurry conveyance system comprising elements 315, 320, 335, 325, 340, 345, and 330 is configured to move slurry from reservoirs (e.g., 305) to the droplet- or stream-producing arrays, as well as recirculate the slurry within the gas-liquid contactor. Comminution systems (320, 325, 330), which may comprise pumps and mixers (e.g., high-shear mixer), provide particle size reduction for the solid component (e.g., proton-removing agents such as Mg(OH)₂, fly ash, cement kiln dust, etc.) of the slurry, thereby improving the participation of the solid in the incorporation of the gas into the liquid. In some embodiments, the comminution systems are screw conveyors in the conduits of the slurry conveyance system (315, 345). In some embodiments, a high-efficiency gas-liquid contactor operably connected to the gas-liquid-solid contactor is employed for removal of additional CO₂ (and criteria pollutants such as SOx) from the CO₂-containing gas stream. The high-efficiency gas-liquid contactor (365) may be configured to produce very fine droplets, thin sheets of liquid, or other high-surface area forms of the divalent-cation containing solution to make efficient contact with the CO₂-containing gas. In some embodiments, the gas-liquid-solid contactor is configured with condensers (370) such that droplets and/or particulates produced by the high-efficiency gas-liquid contactor fall to the reservoir. A gas outlet conduit (375) allows for CO₂-depleted gas to exit the gas-liquid-solid contactor, either to the atmosphere or to another component of the system (e.g., another gas-liquid-contactor). A slurry outlet conduit (380) of the gas-liquid-solid contactor allows for the product slurry comprising a minimal amount of proton-removing agent of the proton-removing agent slurry to leave the gas-liquid-contactor, after which the product slurry is passed to another component of the system such as a precipitating tank, liquid-solid separator, etc.

FIG. 4 provides a horizontally configured gas-liquid contactor or gas-liquid-solid contactor of the invention. In such contactors, a contacting chamber is configured to allow for an immobilization material comprising catalyst, a chamber for physically separating catalyst, or a combination thereof. In such embodiments, the gas-liquid or gas-liquid-solid contactor is configured to allow a CO₂-containing gas to enter through an inlet conduit (400). A divalent cation-containing solution or slurry (additionally comprising one or more proton-removing agents) comprising a liquid and a solid component is allowed to enter the gas-liquid or gas-liquid-solid contactor, respectively, wherein the solution or slurry is passed through an array of droplet- or stream-producing devices (450) (e.g., sprays) to produce sprays of droplets (420) which fill contacting chamber 410 of the contactor. In gas-liquid-solid contactors, proton-removing agent slurry may be subjected to comminution in a comminutor (430) before being provided to contacting chamber 410 of the contactor. In some embodiments, there are at least two sections of droplet production that are operably connected such that CO₂-containing gas travelling the length of the contacting chamber (410) becomes depleted in CO₂ and, if present, criteria pollutants such as SOx. A slurry conveyance system comprising elements 440, 450, 460, 470, 480, and 490 is configured to move product slurry from contacting chamber 410 to other parts of the contactor or to another component of the system. For example, the slurry conveyance system may be configured (as shown) to recirculate the product slurry, moving it to the droplet- or stream-producing devices. As above, comminutor 430 provides particle size reduction for the product slurry during recirculation, thereby improving the participation of the solid (e.g., proton-removing agent such as carbonates) in the incorporation of CO₂ into the liquid. The gas-liquid contactor or gas-liquid-solid contactor is also configured to allow for CO₂-depleted gas to exit the contactor, whereby it is either released to the atmosphere or directed to another component of the system (e.g., another gas-liquid-contactor). A slurry outlet conduit (480) of the gas-liquid-solid contactor allows for the product slurry comprising a minimal amount of proton-removing agent of the proton-removing agent slurry to leave the gas-liquid-contactor, after which the product slurry is passed to another component of the system such as a precipitating tank, liquid-solid separator, etc.

FIG. 5 provides an end-on view of a contactor similarly configured to that of the contactor of FIG. 4. As such, the inlet conduit (500) is configured to allow CO₂-containing gas into contacting chamber 510, within which droplet- or stream-producing devices (550) (e.g., sprays) are configured to produce sprays of droplets (520) that fill the contacting chamber. As described above in relation to FIG. 4, the slurry conveyance system of the gas-liquid or gas-liquid-solid contactor comprises elements 540, 550, 560, 570, 580, and 590 (not shown).

In some embodiments, the invention provides a gas-liquid-solid contactor (601) provided by FIG. 6, which is configured for treating a CO₂-containing gas (610). In some embodiments, the gas-liquid-solid contactor features a contacting chamber (602) comprising a biocatalyst (604) such as an enzyme (e.g., carbonic anhydrase), optionally immobilized as described herein, in suspension in a liquid (603) (e.g., a relatively divalent cation-free solution or a divalent cation-containing solution such as seawater, brine, or an aqueous solution, each optionally including a proton-removing agent etc.), a liquid inlet (605), and liquid (606) and gas (607) outlets in fluid communication with the contacting chamber (602). Gas-liquid or gas-liquid-solid contactors of the invention may advantageously comprise more than one contacting chamber and/or additional liquid and gas conduits (e.g., outlet and inlets). Liquid inlet 605 is for receiving the liquid (603) (e.g., a relatively divalent cation-free solution or a divalent cation-containing solution such as seawater, brine, or an aqueous solution, each optionally including a proton-removing agent etc.) and filling the contacting chamber (602). The contacting chamber (602) is made of an appropriate material that, depending on resources, may be glass, plastic, stainless steel, a synthetic polymer, or another suitable material.

The gas-liquid-solid contactor of FIG. 6 also features a bubbler (608) and a catalyst retainer (609). The bubbler (608) is configured for receiving a CO₂-containing gas (610) to be treated inside the contacting chamber (602) and for bubbling it into the liquid (603), thereby dissolving the CO₂-containing gas (610) in the liquid (603) and creating a pressure within the contacting chamber (602). As above, biocatalysts (604) such as enzymes (e.g., carbonic anhydrase), optionally immobilized as described herein, are chosen so as to be able to efficiently catalyze the hydration of CO₂ and to obtain a treated gas (611) and a solution (612) containing hydrated CO₂ (e.g., bicarbonates and/or carbonates). The liquid outlet (606) is configured for pressure release of solution 612 while catalyst retainer 609 retains the biocatalysts (604), optionally immobilized, within the contacting chamber (602). Gas outlet 607 is configured to release the treated gas (611) from the contacting chamber (602).

The gas-liquid-solid contactor of FIG. 6 further comprises a pressure regulating valve (613) to control pressure created by the CO₂-containing gas (610) bubbled into the contacting chamber (602). As shown, the pressure-regulating valve (613) may be located in the gas outlet (607). The gas-liquid-solid contactor may also include a valve (614) at the liquid outlet (606) and/or at the liquid inlet (605) for regulating the flow of liquid (603) (e.g., a relatively divalent cation-free solution or a divalent cation-containing solution such as seawater, brine, or an aqueous solution, each optionally including a proton-removing agent etc.) into and out of the contacting chamber (602). Such features are advantageous for regulating the pressure inside the contacting chamber (602) so as not to exceed the pressure limits the apparatus may withstand, and for better control the pressure release of the solution (612) containing hydrated CO₂ (e.g., bicarbonates and/or carbonates).

The gas-liquid-solid contactor (601) may further include a mixer within the contacting chamber (602) to mix the liquid (603) (e.g., a relatively divalent cation-free solution or a divalent cation-containing solution such as seawater, brine, or an aqueous solution, each optionally including a proton-removing agent etc.), the biocatalysts (604) (optionally immobilized), and CO₂-containing gas (610). Any type of mixer known in the art may be used. For example, the mixer may comprise an axial propeller operatively connected to a top cover of the contacting chamber (602) by means of a driving shaft. In such a non-limiting example, the gas-liquid-solid contactor further comprises a suitable driving means for driving the shaft into rotation.

As described above, retention of biocatalysts (604) such as enzymes (e.g., carbonic anhydrase), optionally immobilized as described herein, inside the contacting chamber (602) is an important feature of the invention as catalysts, particularly biocatalysts, are often quite expensive. In order to allow the pressure release of hydrated CO₂-containing solution (612) while retaining the biocatalysts (604) (optionally immobilized) within the contacting chamber (602), the catalyst retainer (609) may be adapted according to the relative and respective sizes of the reaction products (e.g., bicarbonates and/or carbonates) and the biocatalysts (604) (e.g., carbonic anhydrase), as well as co-factors when appropriate.

Pressure release of the solution (612) containing CO₂ hydration products may be likened to pressure filtration such as ultrafiltration (i.e., physical separation of particles of 0.005 to 0.1 microns in size) or microfiltration, which is defined as the action of filtering a solution through a fine membrane by pressure.

While the invention may make use of ultrafiltration or microfiltration membranes, it is by no means restricted to their use. For instance, depending upon the size of the biocatalysts (e.g., carbonic anhydrase) and CO₂-hydration products (e.g., bicarbonates and/or carbonates), an appropriate catalyst retainer (609) may comprise a simple grid and/or perforated base at the bottom of the contacting chamber (602) for slowing the flow of solution (612) containing CO₂-hydration products from the contacting chamber (602) while retaining the biocatalysts (604), optionally immobilized as described herein, inside the contacting chamber (602).

The membrane filter may be integrated inside the contacting chamber (602) upstream from the liquid outlet (606). In such embodiments, the liquid flows perpendicularly to the filter as in classic frontal filtration. Appropriate pore size allows permeate liquid (612) (e.g., solution comprising bicarbonates, carbonates, or combinations thereof) to exit through the filter exempt of biocatalysts (604), optionally immobilized biocatalysts. The solution (612) containing the CO₂-hydration products may therefore pass through the filter first in order to be able to exit the contacting chamber (602) via the liquid outlet (606). The permeate liquid (612) (e.g., solution comprising bicarbonates, carbonates, or combinations thereof) or filtrate released may then be passed to another component of the system such as a precipitating tank, liquid-solid separator, etc. Alternatively, the gas-liquid-solid contactor of FIG. 6 may comprise an integrated filter cartridge fixed inside the contacting chamber (602) and positioned at the desired height within the contacting chamber (602). The filter cartridge may be directly linked to the non-pressurized liquid outlet (606) and allow for filtration of the solution containing the CO₂ hydration products (e.g., bicarbonates and/or carbonates), but not the biocatalysts (604) (e.g., carbonic anhydrase) or immobilized biocatalysts, directly into the liquid outlet (606). As mentioned above, the pore size of the membrane inside the cartridge is dependent upon both the size of the biocatalysts (604) (optionally immobilized) and the CO₂ hydration products, as well as co-factors when appropriate. The bubblers of the gas-liquid-solid contactor of FIG. 6 may be in the form of a removable cap (e.g., made of a foam-like material) covering a gas outlet nozzle at the bottom portion of the contacting chamber (602). Foam-like material may be advantageous as it may provide the plurality of gas outlets (628) needed to diffuse very fine bubbles and contribute to their uniform distribution within the liquid (603) (e.g., a relatively divalent cation-free solution or a solution comprising divalent cations such as seawater, brine, or an aqueous solution, each optionally including a proton-removing agent etc.) containing the biocatalysts (604) (e.g., enzymes such as carbonic anhydrase) or immobilized biocatalysts. The reduction in size of the gas bubbles may enhance both gas dissolution and contact surface between CO₂-containing gas (610) and liquid (603) (e.g., solution comprising divalent cations or an aqueous solution optionally including a proton-removing agent) and the biocatalysts (604), optionally immobilized biocatalysts. As stated above, the invention may include a mixer in order to enhance the uniform distribution of CO₂-containing gas (610) bubbles and biocatalysts (604) within the liquid (603) (e.g., a relatively divalent cation-free solution or a divalent cation-containing solution such as seawater, brine, or an aqueous solution, each optionally including a proton-removing agent etc.).

The processor 220 may further include any of a number of different components, including, but not limited to, temperature regulators (e.g., configured to heat the precipitation reaction mixture to a desired temperature); chemical additive components (e.g., for introducing chemical proton-removing agents such as hydroxides, metal oxides, or fly ash); electrochemical components (e.g., cathodes/anodes); components for mechanical agitation and/or physical stirring mechanisms; and components for recirculation of industrial plant flue gas through the precipitation plant. Processor 220 may also contain components configured for monitoring one or more parameters including, but not limited to, internal reactor pressure, pH, precipitation material particle size, metal-ion concentration, conductivity, alkalinity, and pCO₂. Processor 220, in step with the entire precipitation plant, may operate as batch wise, semi-batch wise, or continuously.

Processor 220 may further include an output conveyance for slurry comprising precipitation material or separated supernatant. The processor 220 may also be connected to another container where the aqueous solution of hydrated CO₂ may be treated with the aqueous solution of the divalent cations to form the precipitation material. In some embodiments, the output conveyance may be configured to transport the slurry or supernatant to a tailings pond for disposal or in a naturally occurring body of water, e.g., ocean, sea, lake, or river. In other embodiments, systems may be configured to allow for the slurry or supernatant to be employed as a coolant for an industrial plant by a line running between the precipitation system and the industrial plant. In certain embodiments, the precipitation plant may be co-located with a desalination plant, such that output water from the precipitation plant is employed as input water for the desalination plant. The systems may include a conveyance (i.e., duct) where the output water (e.g., slurry or supernatant) may be directly pumped into the desalination plant.

The system illustrated in FIG. 2 further includes a liquid-solid separator 240 for separating precipitation material from precipitation reaction mixture. The liquid-solid separator may achieve separation of precipitation material from precipitation reaction mixture by draining (e.g., gravitational sedimentation of the precipitation material followed by draining), decanting, filtering (e.g., gravity filtration, vacuum filtration, filtration using forced air), centrifuging, pressing, or any combination thereof. At least one liquid-solid separator is operably connected to the processor such that precipitation reaction mixture may flow from the processor to the liquid-solid separator. As detailed above, any of a number of different liquid-solid separators may be used in combination, in any arrangement (e.g., parallel, series, or combinations thereof), and the precipitation reaction mixture may flow directly to the liquid-solid separator, or the precipitation reaction mixture may be pre-treated.

The system also includes a washer (250) where bulk dewatered precipitation material from liquid-solid separator 240 is washed (e.g., to remove salts and other solutes from the precipitation material), prior to drying at the drying station.

The system may further include a dryer 260 for drying the precipitation material comprising carbonates (e.g., calcium carbonate, magnesium carbonate) and/or bicarbonates produced in the processor. Depending on the particular system, the dryer may include a filtration element, freeze-drying structure, spray-drying structure, or the like. The system may include a conveyer (e.g., duct) from the industrial plant that is connected to the dryer so that a CO₂-containing gas (i.e., industrial plant flue gas) may be contacted directly with the wet precipitation material in the drying stage.

The dried precipitation material may undergo further processing (e.g., grinding, milling) in refining station 270 in order to obtain desired physical properties. One or more components may be added to the precipitation material during refining if the precipitation material is to be used as a building material.

The system further includes outlet conveyers (e.g., conveyer belt, slurry pump) configured for removal of precipitation material from one or more of the following: the processor, dryer, washer, or from the refining station. As described above, precipitation material may be disposed of in a number of different ways. The precipitation material may be transported to a long-term storage site in empty conveyance vehicles (e.g., barges, train cars, trucks, etc.) that may include both above ground and underground storage facilities. In other embodiments, the precipitation material may be disposed of in an underwater location. Any convenient conveyance structure for transporting the precipitation material to the site of disposal may be employed. In certain embodiments, a pipeline or analogous slurry conveyance structure may be employed, wherein these structures may include units for active pumping, gravitational mediated flow, and the like.

A person having ordinary skill in the art will appreciate that flow rates, mass transfer, and heat transfer may vary and may be optimized for systems and methods described herein, and that parasitic load on a power plant may be reduced while carbon sequestration is maximized.

An advantage of systems and methods of the invention, includes, but is not limited to production of carbon-neutral or carbon-negative material through a combination of CO₂ sequestration and CO₂ avoidance, wherein CO₂ avoidance results from, for example, a decrease in production of CO₂-producing cement (e.g., Portland cement). For example, combined CO₂ sequestration and avoidance (e.g., by production of cement, aggregate, etc., as described herein) may result 100-150%, 100-140%, 100-130%, 100-120%, or 100-110% reduction in CO₂. Another advantage of systems and methods of the invention, includes, but is not limited to, capture and sequestration of multiple pollutants from CO₂-containing gas (e.g., flue gas from a coal-fired power plant). For example, SOx (e.g., SO₂) may be absorbed by an alkaline solution comprising divalent cations and converted to sulfite and/or sulfate (e.g., SO₂ absorbed in alkaline liquid, converted to SO₄).

### Compositions

Precipitation material of the invention may comprise several carbonates and/or several carbonate mineral phases resulting from co-precipitation, wherein the precipitation material may comprise, for example, calcium carbonate (e.g., calcite) together with magnesium carbonate (e.g., nesquehonite). Precipitation material may also comprise a single carbonate in a single mineral phase including, but not limited to, calcium carbonate (e.g., aragonite), magnesium carbonate (e.g., nesquehonite), calcium magnesium carbonate (e.g., dolomite). As different carbonates may be precipitated in sequence, the precipitation material may be, depending upon the conditions under which it was obtained, relatively rich (e.g., 90% to 95%) or substantially rich (e.g., 95%-99.9%) in one carbonate and/or one mineral phase, or the precipitation material may comprise an amount of other carbonates and/or other mineral phase (or phases), wherein the desired mineral phase is 50-90% of the precipitation material. It will be appreciated that, in some embodiments, the precipitation material may comprise one or more bicarbonates in addition to the carbonates. It will also be appreciated that, in some embodiments, the precipitation material may comprise one or more hydroxides (e.g., Ca(OH)₂, Mg(OH)₂) in addition to the carbonates. It will also be appreciated that any of the carbonates or hydroxides present in the precipitation material may be wholly or partially amorphous. In some embodiments, the carbonates and/or hydroxides are wholly amorphous.

In some embodiments, the compositions formed from the methods and systems of the invention are metastable carbonate forms such as vaterite, amorphous calcium carbonate (ACC), aragonite, ikaite, a precursor phase of vaterite, a precursor phase of aragonite, an intermediary phase that is less stable than calcite, polymorphic forms in between these polymorphs, and combination thereof. The precursor of vaterite, vaterite, precursor of aragonite, and aragonite can be utilized as a reactive metastable calcium carbonate forms for reaction purposes and stabilization reactions, such as cementing.

The metastable forms such as vaterite and precursor to vaterite and stable carbonate forms such as calcite, may have varying degrees of solubility so that they may dissolve when hydrated in aqueous solutions and reprecipitate stable carbonate minerals, such as calcite. In some embodiments, the reprecipitated form is aragonite.

The compositions of the invention including metastable forms, such as vaterite, surprisingly and unexpectedly are stable compositions in a dry powdered form or in a slurry containing saltwater. The metastable forms in the compositions of the invention may not completely convert to the stable forms, such as calcite, for cementation until contacted with fresh water.

Vaterite may be present in monodisperse or agglomerated form, and may be in spherical, ellipsoidal, plate like shape, or hexagonal system. Vaterite typically has a hexagonal crystal structure and forms polycrystalline spherical particles upon growth. The precursor form of vaterite comprises nanoclusters of vaterite and the precursor form of aragonite comprises sub-micron to nanoclusters of aragonite needles. Aragonite, if present in the composition, may be needle shaped, columnar, or crystals of the rhombic system. Calcite, if present, may be cubic, spindle, or crystals of hexagonal system. An intermediary phase that is less stable than calcite may be a phase that is between vaterite and calcite, a phase between precursor of vaterite and calcite, a phase between aragonite and calcite, and/or a phase between precursor of aragonite and calcite.

In some embodiments, the compositions of the invention include at least 1% vaterite optionally including at least 1% ACC, at least 1% aragonite, and at least 1% calcite, or a combination thereof. In some embodiments, the compositions of the invention include between 1-99% vaterite and optionally, between 1-99% ACC, between 1-99% aragonite, between 1-99% calcite, or a combination thereof.

In some embodiments, the compositions of the invention are hydraulic cement. As used herein, “hydraulic cement” includes a composition which sets and hardens after combining with water or a solution where the solvent is water, e.g., an admixture solution. After hardening, the compositions retain strength and stability even under water. As a result of the immediately starting reactions, stiffening can be observed which may increase with time. After reaching a certain level, this point in time may be referred to as the start of setting. The consecutive further consolidation may be called setting, after which the phase of hardening begins. The compressive strength of the material may then grow steadily, over a period which ranges from a few days in the case of “ultra-rapid-hardening” cements, to several months or years in the case of other cements. Setting and hardening of the product produced by combination of the composition of the invention with an aqueous liquid may or may not result from the production of hydrates that may be formed from the composition upon reaction with water, where the hydrates are essentially insoluble in water. Cements may be employed by themselves or in combination with aggregates, both coarse and fine, in which case the compositions may be referred to as concretes or mortars. Cements may also be cut and chopped to form aggregates.

In some embodiments, the compositions of the invention are supplementary cementitious material. As used herein, “supplementary cementitious material” (SCM) includes SCM as is well known in the art. For example, when SCM of the invention is mixed with Portland cement, one or more properties of that Portland cement after interaction with SCM substantially remain unchanged or are enhanced as compared to the Portland cement itself without SCM or the Portland cement mixed with conventional SCM (such as fly ash). The properties include, but are not limited to, fineness, soundness, consistency, setting time of cement, hardening time of cement, rheological behavior, hydration reaction, specific gravity, loss of ignition, and/or hardness, such as compressive strength of the cement. For example, when 20% of SCM of the invention is added to 80% of OPC (ordinary Portland cement), the one or more properties, such as, for example, compressive strength, of OPC either remain unchanged, decrease by no more than 10%, or are enhanced. The properties of Portland cement may vary depending on the type of Portland cement. The substitution of Portland cement with the SCM of the invention may reduce the CO₂ emissions without compromising the performance of the cement or the concrete as compared to regular Portland cement.

In some embodiments, the composition of the invention after setting, and hardening has a compressive strength of at least 14 MPa; or at least 16 MPa; or at least 18 MPa; or at least 20 MPa; or at least 25 MPa; or at least 30 MPa; or at least 35 MPa; or at least 40 MPa; or at least 45 MPa; or at least 50 MPa; or at least 55 MPa; or at least 60 MPa; or at least 65 MPa; or at least 70 MPa; or at least 75 MPa; or at least 80 MPa; or at least 85 MPa; or at least 90 MPa; or at least 95 MPa; or at least 100 MPa; or from 14-100 MPa; or from 14-80 MPa; or from 14-75 MPa; or from 14-70 MPa; or from 14-65 MPa; or from 14-60 MPa; or from 14-55 MPa; or from 14-50 MPa; or from 14-45 MPa; or from 14-40 MPa; or from 14-35 MPa; or from 14-30 MPa; or from 14-25 MPa; or from 14-20 MPa; or from 14-18 MPa; or from 14-16 MPa; or from 17-35 MPa; or from 17-30 MPa; or from 17-25 MPa; or from 17-20 MPa; or from 17-18 MPa; or from 20-100 MPa; or from 20-90 MPa; or from 20-80 MPa; or from 20-75 MPa; or from 20-70 MPa; or from 20-65 MPa; or from 20-60 MPa; or from 20-55 MPa; or from 20-50 MPa; or from 20-45 MPa; or from 20-40 MPa; or from 20-35 MPa; or from 20-30 MPa; or from 20-25 MPa; or from 30-100 MPa; or from 30-90 MPa; or from 30-80 MPa; or from 30-75 MPa; or from 30-70 MPa; or from 30-65 MPa; or from 30-60 MPa; or from 30-55 MPa; or from 30-50 MPa; or from 30-45 MPa; or from 30-40 MPa; or from 30-35 MPa; or from 40-100 MPa; or from 40-90 MPa; or from 40-80 MPa; or from 40-75 MPa; or from 40-70 MPa; or from 40-65 MPa; or from 40-60 MPa; or from 40-55 MPa; or from 40-50 MPa; or from 40-45 MPa; or from 50-100 MPa; or from 50-90 MPa; or from 50-80 MPa; or from 50-75 MPa; or from 50-70 MPa; or from 50-65 MPa; or from 50-60 MPa; or from 50-55 MPa; or from 60-100 MPa; or from 60-90 MPa; or from 60-80 MPa; or from 60-75 MPa; or from 60-70 MPa; or from 60-65 MPa; or from 70-100 MPa; or from 70-90 MPa; or from 70-80 MPa; or from 70-75 MPa; or from 80-100 MPa; or from 80-90 MPa; or from 80-85 MPa; or from 90-100 MPa; or from 90-95 MPa; or 14 MPa; or 16 MPa; or 18 MPa; or 20 MPa; or 25 MPa; or 30 MPa; or 35 MPa; or 40 MPa; or 45 MPa. For example, in some embodiments of the foregoing aspects and the foregoing embodiments, the composition after setting, and hardening has a compressive strength of 14 MPa to 40 MPa; or 17 MPa to 40 MPa; or 20 MPa to 40 MPa; or 30 MPa to 40 MPa; or 35 MPa to 40 MPa. In some embodiments, the compressive strengths described herein are the compressive strengths after 1 day, or 3 days, or 7 days, or 28 days, or 56 days, or longer.

In some embodiments, the carbon in the vaterite and/or other polymorphs in the composition of the invention, has a δ¹³C of less than −12‰; or less than −13‰; or less than −14‰; or less than −15‰; or less than −16‰; or less than −17‰; or less than −18‰; or less than −19‰; or less than −20‰; or less than −21‰; or less than −22‰; or less than −25‰; or less than −30‰; or less than −40‰; or less than −50‰; or less than −60‰; or less than −70‰; or less than −80‰; or less than −90‰; or less than −100‰; or from −12‰ to −80‰; or from −12‰ to −70‰; or from −12‰ to −60‰; or from −12‰ to −50‰; or from −12‰ to −45‰; or from −12‰ to −40‰; or from −12‰ to −35‰; or from −12‰ to −30‰; or from −12‰ to −25‰; or from −12‰ to −20‰; or from −12‰ to −15‰; or from −13‰ to −80‰; or from −13‰ to −70‰; or from −13‰ to −60‰; or from −13‰ to −50‰; or from −13‰ to −45‰; or from −13‰ to −40‰; or from −13‰ to −35‰; or from −13‰ to −30‰; or from −13‰ to −25‰; or from −13‰ to −20‰; or from −13‰ to −15‰; from −14‰ to −80‰; or from −14‰ to −70‰; or from −14‰ to −60‰; or from −14‰ to −50‰; or from −14‰ to −45‰; or from −14‰ to −40‰; or from −14‰ to −35‰; or from −14‰ to −30‰; or from −14‰ to −25‰; or from −14‰ to −20‰; or from −14‰ to −15‰; or from −15‰ to −80‰; or from −15‰ to −70‰; or from −15‰ to −60‰; or from −15‰ to −50‰; or from −15‰ to −45‰; or from −15‰ to −40‰; or from −15‰ to −35‰; or from −15‰ to −30‰; or from −15‰ to −25‰; or from −15‰ to −20‰; or from −16‰ to −80‰; or from −16‰ to −70‰; or from −16‰ to −60‰; or from −16‰ to −50‰; or from −16‰ to −45‰; or from −16‰ to −40‰; or from −16‰ to −35‰; or from −16‰ to −30‰; or from −16‰ to −25‰; or from −16‰ to −20‰; or from −20‰ to −80‰; or from −20‰ to −70‰; or from −20‰ to −60‰; or from −20‰ to −50‰; or from −20‰ to −40‰; or from −20‰ to −35‰; or from −20‰ to −30‰; or from −20‰ to −25‰; or from −30‰ to −80‰; or from −30‰ to −70‰; or from −30‰ to −60‰; or from −30‰ to −50‰; or from −30‰ to −40‰; or from −40‰ to −80‰; or from −40‰ to −70‰; or from −40‰ to −60‰; or from −40‰ to −50‰; or from −50‰ to −80‰; or from −50‰ to −70‰; or from −50‰ to −60‰; or from −60‰ to −80‰; or from −60‰ to −70‰; or from −70‰ to −80‰; or −12‰; or −13‰; or −14‰; or −15‰; or −16‰; or −17‰; or −18‰; or −19‰; or −20‰; or −21‰; or −22‰; or −25‰; or −30‰; or −40‰; or −50‰; or −60‰; or −70‰; or −80‰; or −90‰; or −100‰. In some embodiments, the composition of the invention includes a CO₂-sequestering additive including carbonates such as vaterite, bicarbonates, or a combination thereof, in which the carbonates, bicarbonates, or a combination thereof have a carbon isotopic fractionation (δ¹³C) value less than −12‰.

The relative carbon isotope composition (δ¹³C) value with units of ‰ (per mille) is a measure of the ratio of the concentration of two stable isotopes of carbon, namely ¹²C and ¹³C, relative to a standard of fossilized belemnite (the PDB standard).

δ¹³C‰=[(¹³C/¹²Cₛₐₘₚₗₑ−¹³C/¹²C_{PDB standard})/(¹³C/¹²C_{PDB standard})]×1000

In some embodiments, when the compositions of embodiments of the invention are derived from a saltwater source, they may include one or more components that are present in the saltwater source which may help in identifying the compositions that come from the saltwater source. These identifying components and the amounts thereof are collectively referred to herein as a saltwater source identifier or “markers.” For example, if the saltwater source is sea water, identifying component that may be present in the composition include, but are not limited to: chloride, sodium, sulfur, potassium, bromide, silicon, strontium and the like. In some embodiments, the composition further includes strontium (Sr). In some embodiments, the Sr is present in the composition in an amount of 1-50,000 parts per million (ppm); or 1-10,000 ppm; or 1-5,000 ppm; or 1-1,000 ppm; or 3-50,000 ppm; or 3-10,000 ppm; or 3-9,000 ppm; or 3-8,000 ppm; or 3-7,000 ppm; or 3-6,000 ppm; or 3-5,000 ppm; or 3-4,000 ppm; or 3-3,000 ppm; or 3-2,000 ppm; or 3-1,000 ppm; or 3-900 ppm; or 3-800 ppm; or 3-700 ppm; or 3-600 ppm; or 3-500 ppm; or 3-400 ppm; or 3-300 ppm; or 3-200 ppm; or 3-100 ppm; or 3-50 ppm; or 3-10 ppm; or 10-50,000 ppm; or 10-10,000 ppm; or 10-9,000 ppm; or 10-8,000 ppm; or 10-7,000 ppm; or 10-6,000 ppm; or 10-5,000 ppm; or 10-4,000 ppm; or 10-3,000 ppm; or 10-2,000 ppm; or 10-1,000 ppm; or 10-900 ppm; or 10-800 ppm; or 10-700 ppm; or 10-600 ppm; or 10-500 ppm; or 10-400 ppm; or 10-300 ppm; or 10-200 ppm; or 10-100 ppm; or 10-50 ppm; or 100-50,000 ppm; or 100-10,000 ppm; or 100-9,000 ppm; or 100-8,000 ppm; or 100-7,000 ppm; or 100-6,000 ppm; or 100-5,000 ppm; or 100-4,000 ppm; or 100-3,000 ppm; or 100-2,000 ppm; or 100-1,000 ppm; or 100-900 ppm; or 100-800 ppm; or 100-700 ppm; or 100-600 ppm; or 100-500 ppm; or 100-400 ppm; or 100-300 ppm; or 100-200 ppm; or 200-50,000 ppm; or 200-10,000 ppm; or 200-1,000 ppm; or 200-500 ppm; or 500-50,000 ppm; or 500-10,000 ppm; or 500-1,000 ppm; or 10 ppm; or 100 ppm; or 200 ppm; or 500 ppm; or 1000 ppm; or 5000 ppm; or 8000 ppm; or 10,000 ppm.

In some embodiments, the composition provided herein is a particulate composition with an average particle size of 0.1-100 microns. The average particle size may be determined using any conventional particle size determination method, such as, but is not limited to, multi-detector laser scattering or sieving (i.e. <38 microns). In certain embodiments, unimodel or multimodal, e.g., bimodal or other, distributions are present. Bimodal distributions allow the surface area to be minimized, thus allowing a lower liquids/solids mass ratio for the cement yet providing smaller reactive particles for early reaction. In such instances, the average particle size of the larger size class can be upwards of 1000 microns (1 mm). In some embodiments, the composition provided herein is a particulate composition with an average particle size of 0.1-1000 microns; or 0.1-900 microns; or 0.1-800 microns; or 0.1-700 microns; or 0.1-600 microns; or 0.1-500 microns; or 0.1-400 microns; or 0.1-300 microns; or 0.1-200 microns; or 0.1-100 microns; or 0.1-90 microns; or 0.1-80 microns; or 0.1-70 microns; or 0.1-60 microns; or 0.1-50 microns; or 0.1-40 microns; or 0.1-30 microns; or 0.1-20 microns; or 0.1-10 microns; or 0.1-5 microns; or 0.5-100 microns; or 0.5-90 microns; or 0.5-80 microns; or 0.5-70 microns; or 0.5-60 microns; or 0.5-50 microns; or 0.5-40 microns; or 0.5-30 microns; or 0.5-20 microns; or 0.5-10 microns; or 0.5-5 microns; or 1-100 microns; or 1-90 microns; or 1-80 microns; or 1-70 microns; or 1-60 microns; or 1-50 microns; or 1-40 microns; or 1-30 microns; or 1-20 microns; or 1-10 microns; or 1-5 microns; or 3-100 microns; or 3-90 microns; or 3-80 microns; or 3-70 microns; or 3-60 microns; or 3-50 microns; or 3-40 microns; or 3-30 microns; or 3-20 microns; or 3-10 microns; or 3-8 microns; or 5-100 microns; or 5-90 microns; or 5-80 microns; or 5-70 microns; or 5-60 microns; or 5-50 microns; or 5-40 microns; or 5-30 microns; or 5-20 microns; or 5-10 microns; or 5-8 microns; or 8-100 microns; or 8-90 microns; or 8-80 microns; or 8-70 microns; or 8-60 microns; or 8-50 microns; or 8-40 microns; or 8-30 microns; or 8-20 microns; or 8-10 microns; or 10-100 microns; or 10-90 microns; or 10-80 microns; or 10-70 microns; or 10-60 microns; or 10-50 microns; or 10-40 microns; or 10-30 microns; or 10-20 microns; or 10-15 microns; or 15-100 microns; or 15-90 microns; or 15-80 microns; or 15-70 microns; or 15-60 microns; or 15-50 microns; or 15-40 microns; or 15-30 microns; or 15-20 microns; or 20-100 microns; or 20-90 microns; or 20-80 microns; or 20-70 microns; or 20-60 microns; or 20-50 microns; or 20-40 microns; or 20-30 microns; or 30-100 microns; or 30-90 microns; or 30-80 microns; or 30-70 microns; or 30-60 microns; or 30-50 microns; or 30-40 microns; or 40-100 microns; or 40-90 microns; or 40-80 microns; or 40-70 microns; or 40-60 microns; or 40-50 microns; or 50-100 microns; or 50-90 microns; or 50-80 microns; or 50-70 microns; or 50-60 microns; or 60-100 microns; or 60-90 microns; or 60-80 microns; or 60-70 microns; or 70-100 microns; or 70-90 microns; or 70-80 microns; or 80-100 microns; or 80-90 microns; or 0.1 microns; or 0.5 microns; or 1 microns; or 2 microns; or 3 microns; or 4 microns; or 5 microns; or 8 microns; or 10 microns; or 15 microns; or 20 microns; or 30 microns; or 40 microns; or 50 microns; or 60 microns; or 70 microns; or 80 microns; or 100 microns. For example, in some embodiments, the composition provided herein is a particulate composition with an average particle size of 0.1-20 micron; or 0.1-15 micron; or 0.1-10 micron; or 0.1-8 micron; or 0.1-5 micron; or 1-5 micron; or 5-10 micron.

In some embodiments, the composition includes one or more different sizes of the particles in the composition. In some embodiments, the composition includes two or more, or three or more, or four or more, or five or more, or ten or more, or 20 or more, or 3-20, or 4-10 different sizes of the particles in the composition. For example, the composition may include two or more, or three or more, or between 3-20 particles ranging from 0.1-10 micron, 10-50 micron, 50-100 micron, 100-200 micron, 200-500 micron, 500-1000 micron, and/or sub-micron sizes of the particles.

In some embodiments, the compositions of the invention are produced without calcination so that minimal emission of CO₂ takes place during the methods and systems of the invention.

While many different carbon-containing salts and compounds are possible due to variability of starting materials, precipitation material comprising magnesium carbonate, calcium carbonate, or combinations thereof is particularly useful. In some embodiments, the precipitation material comprises dolomite (CaMg(CO₃)₂), protodolomite, huntite (CaMg₃(CO₃)₄), and/or sergeevite (Ca₂Mg₁₁(CO₃)₁₃.H₂O), which are carbonate minerals comprising both calcium and magnesium.

In some embodiments, the precipitation material comprises calcium carbonate in one or more phases selected from calcite, aragonite, vaterite, or a combination thereof. In some embodiments, the precipitation material comprises hydrated forms of calcium carbonate selected from ikaite (CaCO₃.6H₂O), amorphous calcium carbonate (CaCO₃.nH₂O), monohydrocalcite (CaCO₃.H₂O), or combinations thereof. In some embodiments, the precipitation material comprises magnesium carbonate, wherein the magnesium carbonate does not have a water of hydration. In some embodiments, the precipitation material comprises magnesium carbonate, wherein the magnesium carbonate may have any of a number of different waters of hydration selected from 1, 2, 3, 4, or more than 4 waters of hydration. In some embodiments, the precipitation material comprises 1, 2, 3, 4, or more than 4 different magnesium carbonate phases, wherein the magnesium carbonate phases differ in the number of waters of hydration. For example, precipitation material may comprise magnesite (MgCO₃), barringtonite (MgCO₃.2H₂O), nesquehonite (MgCO₃.3H₂O), lansfordite (MgCO₃.5H₂O), and amorphous magnesium carbonate. In some embodiments, precipitation material comprises magnesium carbonates that include hydroxide and waters of hydration such as artinite (MgCO₃.Mg(OH)₂.3H₂O), hydromagnesite (Mg₅(CO₃)₄(OH)₂.3H₂O), or combinations thereof. As such, precipitation material may comprise carbonates of calcium, magnesium, or combinations thereof in all or some of the various states of hydration listed herein.

Precipitation material may be in a storage-stable form (which may simply be air-dried precipitation material), and may be stored above ground under exposed conditions (i.e., open to the atmosphere) without significant, if any, degradation for extended durations. In some embodiments, the precipitation material is stable under exposed conditions for 1 year or longer, 5 years or longer, 10 years or longer, 25 years or longer, 50 years or longer, 100 years or longer, 250 years or longer, 1000 years or longer, 10,000 years or longer, 1,000,000 years or longer, or even 100,000,000 years or longer. The aboveground storage-stable forms of the precipitation material are stable under a variety of different environment conditions, e.g., from temperatures ranging from −100° C. to 600° C. and humidity ranging from 0 to 100% where the conditions may be calm, windy, or stormy. As the storage-stable form of the precipitation material undergoes little if any degradation while stored above ground under normal rainwater pH, the amount of degradation, if any, as measured in terms of CO₂ gas release from the product, does not exceed 5% per year, and in certain embodiments will not exceed 1% per year. Indeed, precipitation material provided by the invention does not release more than 1%, 5%, or 10% of its total CO₂ when exposed to normal conditions of temperature and moisture, including rainfall of normal pH for at least 1, 2, 5, 10, or 20 years, or for more than 20 years, for example, for more than 100 years. In some embodiments, the precipitation material does not release more than 1% of its total CO₂ when exposed to normal conditions of temperature and moisture, including rainfall of normal pH for at least 1 year. In some embodiments, the precipitation material does not release more than 5% of its total CO₂ when exposed to normal conditions of temperature and moisture, including rainfall of normal pH for at least 1 year. In some embodiments, the precipitation material does not release more than 10% of its total CO₂ when exposed to normal conditions of temperature and moisture, including rainfall of normal pH for at least 1 year. In some embodiments, the precipitation material does not release more than 1% of its total CO₂ when exposed to normal conditions of temperature and moisture, including rainfall of normal pH for at least 10 years. In some embodiments, the composition does not release more than 1% of its total CO₂ when exposed to normal conditions of temperature and moisture including rainfall of normal pH for at least 100 years. In some embodiments, the precipitation material does not release more than 1% of its total CO₂ when exposed to normal conditions of temperature and moisture, including rainfall of normal pH for at least 1000 years.

Any suitable surrogate marker or test that is reasonably able to predict such stability may be used. For example, an accelerated test comprising conditions of elevated temperature and/or moderate to more extreme pH conditions is reasonably able to indicate stability over extended periods of time. For example, depending on the intended use and environment of the precipitation material, a sample of the precipitation material may be exposed to 50, 75, 90, 100, 120, or 150° C. for 1, 2, 5, 25, 50, 100, 200, or 500 days at between 10% and 50% relative humidity, and a loss less than 1%, 2%, 3%, 4%, 5%, 10%, 20%, 30%, or 50% of its carbon may be considered sufficient evidence of stability of materials of the invention for a given period (e.g., 1, 10, 100, 1000, or more than 1000 years).

Any of a number of suitable methods may be used to test the stability of the precipitation material including physical test methods and chemical test methods, wherein the methods are suitable for determining that the compounds in the precipitation material are similar to or the same as naturally occurring compounds known to have the above specified stability (e.g., limestone). CO₂ content of the precipitation material may be monitored by any suitable method, one such non-limiting example being coulometry. Other conditions may be adjusted as appropriate, including pH, pressure, UV radiation, and the like, again depending on the intended or likely environment. It will be appreciated that any suitable conditions may be used that one of skill in the art would reasonably conclude indicate the requisite stability over the indicated time period. In addition, if accepted chemical knowledge indicates that the precipitation material would have the requisite stability for the indicated period this may be used as well, in addition to or in place of actual measurements. For example, some carbonate compounds that may be part of a precipitation material of the invention (e.g., in a given polymorphic form) may be well-known geologically and known to have withstood normal weather for decades, centuries, or even millennia, without appreciable breakdown, and so have the requisite stability. The aboveground storage-stable forms of the precipitation material are stable under a variety of different environment conditions, e.g., from temperatures ranging from −100° C. to 600° C. and humidity ranging from 0 to 100% where the conditions may be calm, windy, or stormy.

### EXAMPLES

### Example 1

### Carbonic Anhydrase

Experiments were carried out in a 1-L gas-liquid contactor/reactor in semi-batch mode using about 1 L liquid volume, 1.5 SLPM (standard liters per minute) 15% CO₂, and 10% NaOH (sodium hydroxide) (weight/volume, w/v)) for pH control. A relay on a pH controller was used to actuate a dosing pump for 10% NaOH (w/v) and maintain target pH values. Different carbonic anhydrase mass loadings (e.g., 0 mg/L, 0.1 mg/L, 1 mg/L, 10 mg/L) at different pH levels (e.g., pH 8, pH 10) were used to illustrate the effects of changing these variables. In addition, calcium chloride was used as a bicarbonate sink to illustrate the enhancement in CO₂ absorption.

### Materials and Equipment

ThermoScientific CO₂ analyzer (with LabView software)
1 L gas-liquid contactor/reactor and ancillary equipment for mass flow control
Carbonic anhydrase (extracted from bovine erythrocytes and freeze dried; available from VWR)
Deionized water
10% NaOH (w/v) (for pH control)
Eppendorf pipettes
Calcium chloride

### Procedure

1. The gas-liquid contactor/reactor was plumbed for gas outlet monitoring via the CO₂ analyzer.
2. The pH meter was calibrated and the probe was inserted in the gas-liquid contactor/reactor.
3. LabView software was started for CO₂ analyzer and pH probe data logging.
4. The caustic dosing pump was connected to the pH controller and the caustic reservoir was filled with 10% NaOH (aq) (w/v).
5. Using a graduated cylinder, 1-L of deionized water was measured out.
6. The carbonic anhydrase was added to the deionized water and was mixed.
7. The head mixer was started to keep contents suspended.
8. Using mass flow controllers, 1.5 SLPM of 15% CO₂ was sparged.
9. The CO₂ absorption and pH was monitored.

### Results

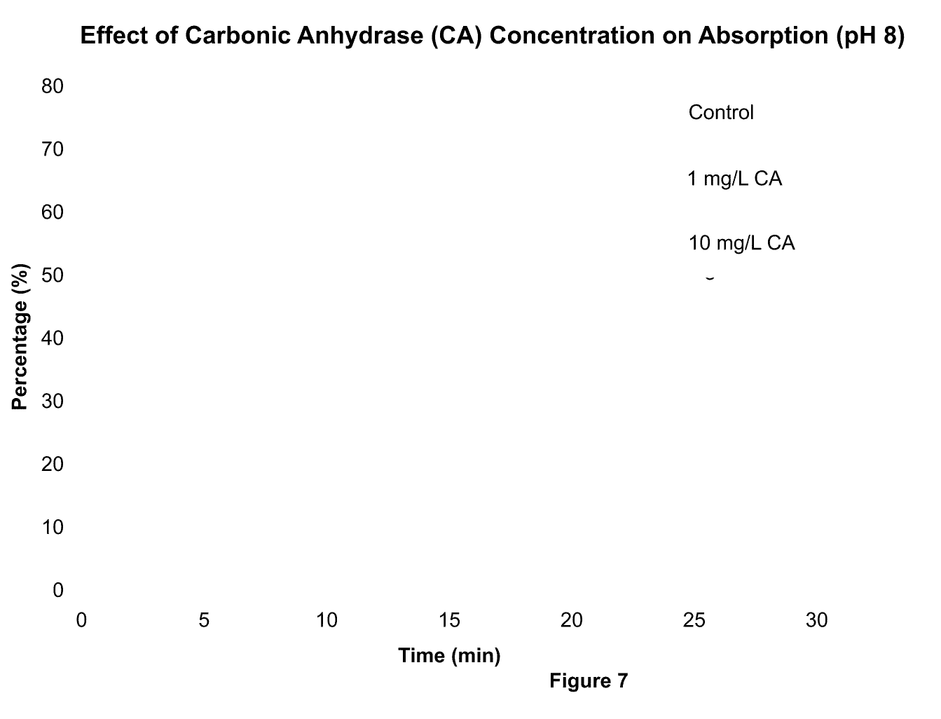

FIG. 7 illustrates a plot of the effect of carbonic anhydrase (CA) concentration at 1 mg/L and 10 mg/L on % absorption of carbon dioxide at pH 8. The control was a basic solution with no CA. Both 1 mg/L and 10 mg/L showed saturation of the CO₂ absorption between 50-60%.

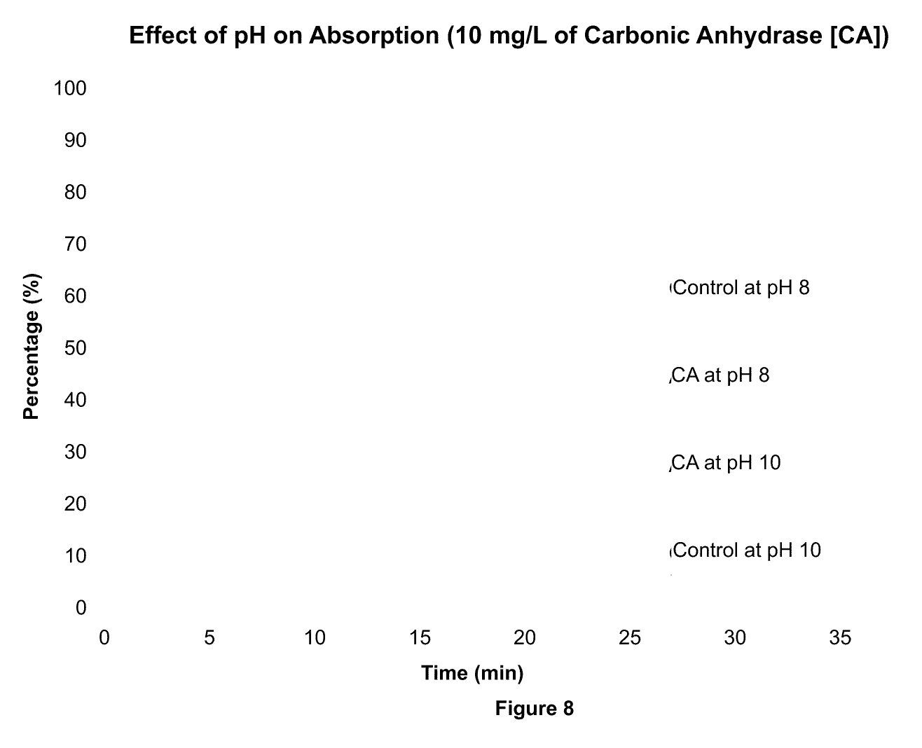

FIG. 8 illustrates a plot of the effect of pH at pH 8 and pH 10 on % absorption of carbon dioxide at 10 mg/L of carbonic anhydrase. CA at pH 10 showed between 70-80% absorption of CO₂.

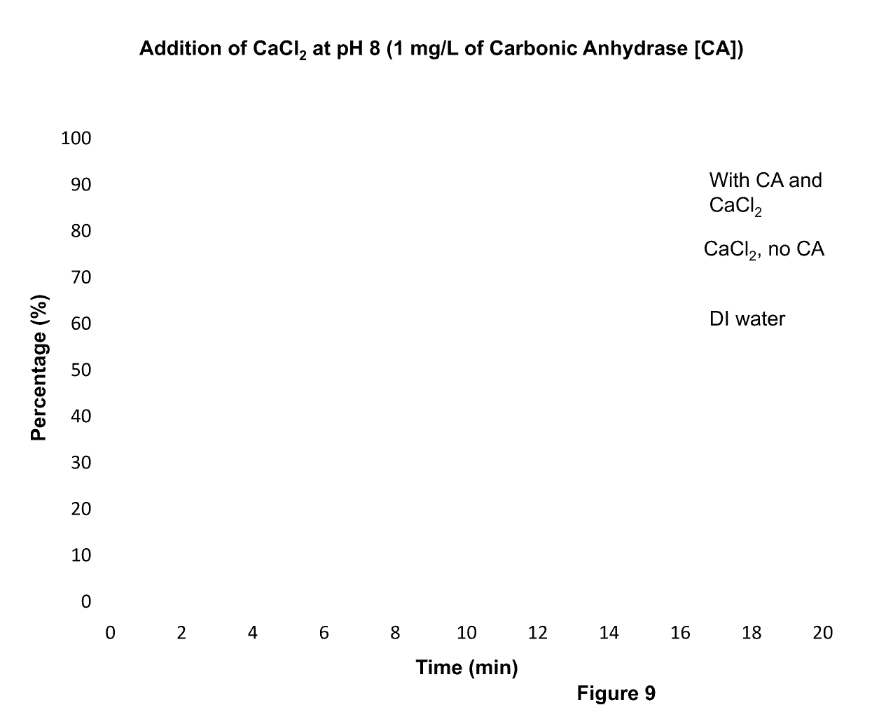

FIG. 9 illustrates a plot of the effect of calcium chloride (70% w/v) on the % absorption of carbon dioxide at pH 8 and 1 mg/L of carbonic anhydrase. CA with CaCl₂ showed between 80-90% CO₂ absorption.

The data illustrates that the factors such as catalyst, the higher pH and the removal of the carbonate from the solution, enhance the absorption of the carbon dioxide into the solution,

While preferred embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein might be employed in practicing the invention. It is intended that the following claims define the scope of the invention and that methods and structures within the scope of these claims and their equivalents be covered thereby.

1. A method, comprising:
(i) contacting a gaseous stream comprising CO₂ with a catalyst to form a solution comprising hydrated CO₂; and
(ii) treating the solution to produce a composition comprising a metastable carbonate.

2. The method of claim 1, wherein the metastable carbonate is more stable in salt water than in fresh water.

3. The method of claim 1, wherein the metastable carbonate is selected from the group consisting of vaterite, aragonite, amorphous calcium carbonate, and combination thereof.

4. The method of claim 1, wherein treating the solution comprises treating the solution comprising hydrated CO₂ with an aqueous solution comprising divalent cations.

5. The method of claim 1, wherein the composition comprises calcium carbonate, magnesium carbonate, calcium magnesium carbonate, or a combination thereof.

6. The method of claim 1, wherein the composition is further treated to produce a dry particulate composition.

7. The method of claim 6, wherein the dry particulate composition has an average particle size of 0.1 to 100 microns.

8. The method of claim 6, wherein the dry particulate composition is incorporated into a cement or concrete composition.

9. The method of claim 8, wherein the concrete composition further comprises ordinary Portland cement, aggregate, admixture, or a combination thereof.

10. The method of claim 8, wherein the cement or concrete composition upon combination with water, setting, and hardening has a compressive strength in a range of 20-70 MPa.

11. The method of claim 1, wherein the gaseous stream comprises a waste stream or product from an industrial plant selected from power plant, chemical processing plant, or other industrial plant that produces CO₂ as a byproduct.

12. The method of claim 1, wherein the catalyst is an enzyme.

13. The method of claim 1, wherein treating the solution to produce a composition comprising a metastable carbonate comprises treating the solution with a proton-removing agent.

14. The method of claim 1 wherein treating the solution to produce a composition comprising a metastable carbonate comprises separating the catalyst from the solution.

15. The method of claim 1, further comprising producing a building material from the composition comprising the metastable carbonate.

16. A method, comprising:
(i) contacting a gaseous stream comprising CO₂ with a catalyst to form a solution comprising hydrated CO₂;
(ii) treating the solution with a proton-removing agent; and
(ii) injecting the solution underground.

17. The method of claim 16, wherein the catalyst is a biocatalyst.

18. The method of claim 16, wherein the biocatalyst is carbonic anhydrase.

19. The method of claim 16, wherein treating the solution with a proton-removing agent comprises treating the solution with an electrochemically produced proton-removing agent.

20. The method of claim 16, wherein the proton-removing agent is sodium hydroxide.

21. The method of claim 20, wherein the sodium hydroxide is electrochemically produced without producing chlorine gas at the anode.

22. The method of claim 20, wherein the sodium hydroxide is electrochemically produced without producing oxygen gas at the anode.

23. The method of claim 16, wherein injecting the solution underground comprises injecting the solution into a saline aquifer, a petroleum reservoir, a deep coal seem, a sub-oceanic formation, or some combination thereof.

24. The method of claim 23, wherein injecting the solution underground comprises injecting the solution into a saline aquifer.

25. The method of claim 24, wherein the capacity of the saline aquifer is increased prior to injecting the solution into the saline aquifer, wherein increasing the capacity of the saline aquifer comprises removing aquifer water.

26. (canceled)

27. A composition, comprising an immobilized catalyst on immobilization material, a substrate of the catalyst, a product of the catalyst, and water.

28. The composition of claim 27, wherein the catalyst is carbonic anhydrase, the substrate is dissolved CO₂, and the product is bicarbonate.

29. The composition of claim 28, wherein the immobilization material selected from alumina; bentonite; a biopolymers; calcium carbonate; calcium phosphate; carbon; a ceramic support; a clay; a porous metal structure; collagen; glass; hydroxyapatite; an ion-exchange resin; kaolin; a polymer mesh; a polysaccharide; a phenolic polymer; polyaminostyrene; polyacrylamide; poly(acryloyl morpholine); polypropylene; a polymer hydrogel; sephadex; sepharose; a treated silicon oxide; silica gel; and PTFE (polytetrafluoroethylene).

30. The composition of claim 29, further comprising dissolved SOx, dissolved NOx, one or more dissolved mercury salts, or some combination thereof.

31. The composition of claim 30, wherein the dissolved SOx comprises sulfite, sulfate, or a combination thereof.

32. The composition of claim 30, wherein the dissolved NOx comprises nitrite, nitrate, or a combination thereof.

33. A system comprising:
a) a source of CO₂;
b) a processor comprising a catalyst adapted to produce a solution comprising hydrated CO₂, wherein the processor is operably connected to the source of CO₂; and
c) a reactor configured to produce a composition comprising a metastable carbonate.

34. The system of claim 33, further comprising a source of divalent cations operably connected to the processor and/or the reactor.

35. The system of claim 33, wherein the catalyst is immobilized in the processor.

36. The system of claim 35, wherein the catalyst is part of an immobilization material selected from alumina; bentonite; a biopolymers; calcium carbonate; calcium phosphate; carbon; a ceramic support; a clay; a porous metal structure; collagen; glass; hydroxyapatite; an ion-exchange resin; kaolin; a polymer mesh; a polysaccharide; a phenolic polymer; polyaminostyrene; polyacrylamide; poly(acryloyl morpholine); polypropylene; a polymer hydrogel; sephadex; sepharose; a treated silicon oxide; silica gel; and PTFE (polytetrafluoroethylene).

37. The system of claim 33, wherein the processor comprises a gas-liquid contactor.

38. The system of claim 33, wherein the processor comprises a gas-liquid-solid contactor.
